SEQUENCE LISTING (27 sequences)

SEQ ID NO: 1 - protein, 10 aa, synthetic construct
  REGION 1..10: note = Anti TfR HCDR1
GFTFSSYAMN

SEQ ID NO: 2 - protein, 10 aa, synthetic construct
  REGION 1..10: note = Anti TfR HCDR2
GISGSGGHTY

SEQ ID NO: 3 - protein, 13 aa, synthetic construct
  REGION 1..13: note = Anti TfR HCDR3
EGYDSSGYNPFDY

SEQ ID NO: 4 - protein, 11 aa, synthetic construct
  REGION 1..11: note = Anti TfR LCDR1
SGDKLGDKYAS

SEQ ID NO: 5 - protein, 7 aa, synthetic construct
  REGION 1..7: note = Anti TfR LCDR2
QDSKRPS

SEQ ID NO: 6 - protein, 9 aa, synthetic construct
  REGION 1..9: note = Anti TfR LCDR3
QAWDSSTVV

SEQ ID NO: 7 - protein, 246 aa, synthetic construct
  REGION 1..246: note = Anti-TfR scFv
EVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVL

SEQ ID NO: 8 - protein, 5 aa, synthetic construct
  REGION 1..5: note = Anti-pyroglutamate amyloid-b HCDR1
SYDMY

SEQ ID NO: 9 - protein, 17 aa, synthetic construct
  REGION 1..17: note = Anti-pyroglutamate amyloid-b HCDR2
YIDSDSGDTSYNQKFKG

SEQ ID NO: 10 - protein, 7 aa, synthetic construct
  REGION 1..7: note = Anti-pyroglutamate amyloid-b HCDR3
YRYAMDY

SEQ ID NO: 11 - protein, 16 aa, synthetic construct
  REGION 1..16: note = Anti-pyroglutamate amyloid-b LCDR1
KSSQSLLDSRAKTYLT

SEQ ID NO: 12 - protein, 7 aa, synthetic construct
  REGION 1..7: note = Anti-pyroglutamate amyloid-b LCDR2
LVSKLDS

SEQ ID NO: 13 - protein, 9 aa, synthetic construct
  REGION 1..9: note = Anti-pyroglutamate amyloid-b LCDR3
WQGTHFPYT

SEQ ID NO: 14 - protein, 116 aa, synthetic construct
  REGION 1..116: note = Anti-pyroglutamate amyloid-b VH
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSS

SEQ ID NO: 15 - protein, 112 aa, synthetic construct
  REGION 1..112: note = Anti-pyroglutamate amyloid-b VL
DVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGQGTKLEIK

SEQ ID NO: 16 - protein, 10 aa, synthetic construct
  REGION 1..10: note = Anti-pyroglutamate amyloid-b HCDR1
GHVFTSYDMY

SEQ ID NO: 17 - protein, 10 aa, synthetic construct
  REGION 1..10: note = Anti-pyroglutamate amyloid-b HCDR2
YIDSDSGDTS

SEQ ID NO: 18 - DNA, 3543 bp, synthetic construct
  misc_feature 1..3543: note = Heavy Chain 1
  source 1..3543: mol_type = other DNA
atggccaggaagtccgctctgctcgctctggcacttctgcttctgggatttggacctgcttgggctgacatcgtgatgacccagacacctctgagcagccccgttacattgggccagcctgcctccatctcctgccggtcctctaagtccctgctgtacaaggacggcaagacctacctgaactggctgcagcagaggcctggctgtcctcctagactgctgatctacctgatgtccaccagagcctctggcgtgcccgatagattttctggctctggcgctggcaccgacttcaccctgaagatctctagagtggaagccgaggacgtgggcgtgtactactgtcagcagctggtggactaccctctgacctttggccagggcaccaagctggaaatcaagggcggaggttctggtggatctggcggatgtcctccttgtggtggaagcggcggacaggttcagctggttcagtctggagccgaagtgaagaaacctggcgcctctgtcaaggtgtcctgcaaggcttccggctacacctttaccagctactggatcacctgggtccgacagaggcctggacaaggactcgaatggatgggcgacatccatcctggcagaggctccactaagtacgcccagaaactgcagggcagagtgaccatgaccaccgacacctctacctccaccgcctacatggaactgcggtccctgagatctgacgacaccgccgtgtactactgcgccagaagatggggcttcgactactggggctgtggcacactggtcaccgtttcttctgagcccaaatctagcgacaaaactcacacatgcccaccgtgcccagcacctgaagccgccgggggaccgtcagtcttcctcttccccccaaaacccaaggacaccctctacatcacccgggagcctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcatggccaggaagtccgctctgctcgctctggcacttctgcttctgggatttggacctgcttgggctcaggttcagctggttcagtctggagccgaagtgaagaaacctggcgcctctgtgaaggtgtcctgcaaggcttctggccacgtgttcaccagctacgacatgtactgggtccgacaggctcctggacagggacttgagtggatcggctacatcgactccgactccggcgacacctcctacaaccagaaattcaagggcagagtgaccctgaccgtggacacctctacctccaccgtgtacatggaactgtccagcctgagatccgaggacaccgccgtgtactactgcgcctactacagatacgccatggactactggggccagggcacactggttaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctctacatcacccgggagcctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaaggcggagctggtggtgctgaggttcagctgctcgagtctggcggaggattggttcagcctggcggctctctgagactgtcttgtgccgactccggcttcaccttctccagctacgccatgaactgggtccgacaggctcctggctgtggactggaatgggtgtccggcatttctggctctggcggccacacctactacgccgattctgtgaagggcagattcaccgtgtctcgggacaactccaagaacaccctgtacctgcagatgaactccctgagagccgaggacaccgccgtgtactactgtgctagagagggctacgactcctccggctacaacccctttgattactggggccagggcacccaagtgaccgtttcttctggcggaggttctggtggatctggcggatgtcctccttgtggtggaagcggcggatcctacgagctgacccagcctccttccgtgtctgtgtctcctggccagaccgcctccatcacctgttctggcgataagctgggcgataagtacgcctcctggtatcagcagaagcccggccagtctcctgtgctggtcatctaccaggactccaagcggccttctggcatccctgagagattctccggctccaactccggcaataccgccacactgaccatctctggcacacaggccatggacgaggccgactactattgtcaggcctgggactcctccaccgtggtgtttggctgtggcaccaagctgacagtgctgcac

SEQ ID NO: 19 - DNA, 723 bp, synthetic construct
  misc_feature 1..723: note = Light chain
  source 1..723: mol_type = other DNA
atggccaggaagtccgctctgctcgctctggcacttctgcttctgggatttggacctgcttgggctgacgtggtcatgacacagagccctctgagcctgcctgtgacattgggacagcctgcctccatctcctgcaagtcctctcagtccctgctggactccagagccaagacctatctgacctggctgcagcagaggcctggccagtctcctagaaggctgatctacctggtgtccaagctggactctggcgtgcccgatagattctccggctctggctctggcaccgacttcaccctgaagatctccagagtggaagccgaggacgtgggcgtgtactactgttggcagggcacccactttccatacaccttcggccagggcacaaagctggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - DNA, 1404 bp, synthetic construct
  misc_feature 1..1404: note = Heavy Chain 2
  source 1..1404: mol_type = other DNA
atggccaggaagtccgctctgctcgctctggcacttctgcttctgggatttggacctgcttgggctcaggttcagctggttcagtctggagccgaagtgaagaaacctggcgcctctgtgaaggtgtcctgcaaggcttctggccacgtgttcaccagctacgacatgtactgggtccgacaggctcctggacagggacttgagtggatcggctacatcgactccgactccggcgacacctcctacaaccagaaattcaagggcagagtgaccctgaccgtggacacctctacctccaccgtgtacatggaactgtccagcctgagatccgaggacaccgccgtgtactactgcgcctactacagatacgccatggactactggggccagggcacactggttaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctctacatcacccgggagcctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 21 - protein, 721 aa, synthetic construct
  REGION 1..721: note = Heavy Chain 1 w/signal peptide
MARKSALLALALLLLGFGPAWAQVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGAGGAEVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVLH

SEQ ID NO: 22 - protein, 241 aa, synthetic construct
  REGION 1..241: note = Light chain w/signal peptide
MARKSALLALALLLLGFGPAWADVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 468 aa, synthetic construct
  REGION 1..468: note = Heavy chain 2 w/signal peptide
MARKSALLALALLLLGFGPAWAQVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 699 aa, synthetic construct
  REGION 1..699: note = Heavy chain 1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGAGGAEVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVLH

SEQ ID NO: 25 - protein, 219 aa, synthetic construct
  REGION 1..219: note = Light chain
DVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 446 aa, synthetic construct
  REGION 1..446: note = Heavy chain 2
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 18 aa, synthetic construct
  REGION 1..18: note = Linker sequence
GGGSGGSGGCPPCGGSGG

(19) United States
(12) Patent Application Publication
(10) Pub. No.: US 2025/0188181 A1
(43) Pub. Date: Jun. 12, 2025

(54) MULTISPECIFIC ANTIBODIES AND USES THEREOF

(71) Applicant: Janssen Pharmaceutica NV, Beerse (BE)

(72) Inventors: Sanjaya SINGH, Blue Bell, PA (US); Danlin YANG, Philadelphia, PA (US); Bidisha DASGUPTA, Spring House, PA (US); Rupesh NANJUNDA, Malvern, PA (US); Adam ZWOLAK, Spring House, PA (US); Wilson EDWARDS, San Diego, CA (US); Brian DEL ROSARIO, Spring House, PA (US); Thomas KELLY, Spring House, PA (US)

(21) Appl. No.: 18/846,006

(22) PCT Filed: Mar. 10, 2023
(86) PCT No.: PCT/EP2023/056213

§ 371 Date: Sep. 11, 2024

Related U.S. Application Data
(60) Provisional application No. 63/269,204, filed on Mar. 11, 2022.

(51) Int. Cl. C07K 16/28; C07K 16/18

(52) U.S. Cl. CPC C07K 16/2881; C07K 16/18; C07K 2317/31; C07K 2317/522; C07K 2317/524; C07K 2317/526; C07K 2317/565; C07K 2317/622; C07K 2317/71; C07K 2317/72

(57) ABSTRACT

Provided are multispecific antibodies or antigen binding fragments thereof comprising at least one first antigen-binding region capable of binding specifically to pyroglutamate amyloid-β and a second antigen-binding region capable of binding specifically to transferrin receptor (TfR). Also provided are methods of treating or detecting a neurological disorder and/or delivering a therapeutic or diagnostic agent across the blood-brain barrier. Also described are nucleic acids encoding the antibodies, vectors comprising the nucleic acids, recombinant host cells comprising the nucleic acids and/or vectors, and methods of producing the multispecific antibodies or antigen binding fragments thereof.

### CROSS REFERENCE TO RELATED APPLICATION

This application claims the benefit of U.S. Provisional Patent Application No. 63/269,204, filed Mar. 11, 2022, the disclosure of which is incorporated by reference herein in entirety.

### REFERENCE TO SEQUENCE LISTING SUBMITTED ELECTRONICALLY

This application contains a sequence listing, which is submitted electronically. The content of the electronic sequence listing (065768-121WO1_Sequence Listing.xml; size: 43,618 bytes; and date of creation: Mar. 2, 2023) is herein incorporated by reference in its entirety.

### FIELD OF THE INVENTION

The application relates to anti-pyroglutamate amyloid-3/anti-transferrin receptor (TfR) antibodies, antibody conjugates, nucleic acids and expression vectors encoding the antibodies, recombinant cells containing the expression vectors, and compositions comprising the antibodies. Methods of making the antibodies, methods of using the antibodies to treat conditions including neurological disorders (e.g., amyloid-related disorders), and methods of using the antibodies to diagnose neurological disorders are also provided.

### BACKGROUND OF THE INVENTION

Alzheimer's disease (AD) is a degenerative brain disorder characterized clinically by progressive loss of memory, cognition, reasoning, judgment and emotional stability that gradually leads to profound mental deterioration and ultimately death. Alzheimer's disease is a common cause of progressive mental failure (dementia) in the elderly. Alzheimer's disease has been observed worldwide and represents a major public health issue. The disease is currently estimated to affect more than five million individuals in the United States alone. At present it is incurable, and no treatment effectively prevents AD or reverses its symptoms or course.

The brains of individuals with AD exhibit characteristic lesions termed amyloid plaques, amyloid angiopathy (amyloid deposits in blood vessels) and neurofibrillary tangles. Large numbers of these lesions, particularly amyloid plaques and neurofibrillary tangles, are generally found in several areas of the brain important for memory and cognitive function. Amyloid plaques and amyloid angiopathy also characterize the brains of individuals with Trisomy 21 (Down's Syndrome), diffuse Lewy body disease and hereditary cerebral hemorrhage with amyloidosis of the Dutch-type (HCHWA-D).

A major constituent of amyloid plaques is a variety of amyloid-beta (Aβ) peptides that are produced by cleavage of the β-amyloid precursor protein (APP). Deposition of Aβ peptides in brain is hypothesized to be an early and necessary step in the disease cascade leading to AD. The identification of mutations in the amyloid precursor protein and presenillin genes resulting in altered Aβ production and causing familial early onset AD provides strong evidence that altered amyloid metabolism is a central event in the pathogenic process underlying the disease.

Amyloid-β peptides having pyroglutamate at the third residue (3pE Aβ) are a major species deposited in the brain of AD patients. 3pE Aβ is present in almost all diffuse and mature plaques in AD, is metabolically stable, and can play a role in both plaque seeding and stabilization (Cynis et al., Molecular Neurodegeneration, 2016; 11:48). Detectable amounts of 3pE Aβ have not been reported in CSF or plasma, thus suggesting that the target peptide is pathology specific (DeMattos et al., Neuron, 2012; 76:1-13). Antibodies that selectively bind to 3pE Aβ can be useful for immunotherapy.

While the blood-brain barrier (BBB) prevents harmful substances from entering the brain and is essential for brain homeostasis, it presents a formidable obstacle for efficiently delivering drugs to the brain. Large molecules, such as monoclonal antibodies and other biotherapeutics, have great therapeutic/diagnostic potential for treating/detecting pathology in the central nervous system (CNS). However, their route into the brain is prevented by the BBB. Previous studies have illustrated that only a very small percentage (approximately 0.1%) of an IgG injected in the bloodstream are able to penetrate the BBB into the CNS compartment (Felgenhauer, Klin. Wschr. 52: 1158-1164, 1974)). This will limit any pharmacological effect due to the low concentration within the CNS of the antibody.

Numerous approaches have been studied to improve the brain delivery of therapeutic monoclonal antibodies (mAbs), including the use of receptor-mediated transcytosis (RMT). RMT utilizes abundantly expressed receptors on the luminal side of the BBB for transport through brain endothelial cells. Previous efforts to generate a clinically feasible platform for delivery of therapeutic mAbs into the brain have been focused on antibody engineering to increase the efficiency of transcytosis, with gains made through observations on valency of binding, pH dependency and affinity (reviewed in Goulatis et al., 2017, Curr Opin Struct Biol 45: 109-115). However, translation into NHPs and the clinic has been limited by rapid peripheral clearance from target-mediated drug disposition (TMDD) and safety from acute reticulocyte depletion (Gadkar, 2016, Eur J Pharm Biopharm. 2016 April; 101:53-61). Transferrin receptor (TfR), particularly TfR1, mediates the transport of iron-loaded transferrin (Tf) from blood to brain and the return of iron-depleted Tf to the blood (Kawabata, Free Radical Biology & Medicine, 133, 46-54, 2019). Anti-TfR1 monoclonal antibodies have been used to deliver drugs to the brain (Burkhart, et al. Progress in neurobiology, 181, 101665, 2019). However, safety liabilities and poor pharmacokinetics (PK) of anti-TfR1 monoclonal antibodies have hampered their clinical development as BBB carriers.

Therefore, there is a need for an anti-pyroglutamate amyloid-β/anti-TfR antibody or antigen binding fragment thereof that can be used to cross the BBB and target 3pE Aβ for immunotherapy.

### SUMMARY OF THE INVENTION

As embodied and fully described, the invention relates to multispecific antibodies and antigen binding fragments thereof that bind to amyloid-β having pyroglutamate at the third residue (3pE Aβ) and bind to transferrin receptor (TfR), methods of producing multispecific antibodies or antigen binding fragments thereof that bind to 3pE Aβ and TfR, assay methods using such multispecific antibodies or antigen binding fragments thereof, and use of the multispecific antibodies or antigen binding fragments thereof of the invention for the manufacture of a medicament, for treating, delaying the onset of or reversing at least one pathology or symptom of a neurological disorder, such as, for example, Alzheimer's disease and other β-amyloid-related diseases.

In one general aspect, the application relates to a multispecific antibody or antigen binding fragment thereof comprising at least one first antigen-binding region capable of binding specifically to pyroglutamate amyloid-β and a second antigen-binding region capable of binding specifically to transferrin receptor (TfR). In certain embodiments, (a) the first antigen-binding region comprises (i) a first heavy chain variable region (VH1) comprising heavy chain complementarity determining region 1 (HCDR1), HCDR2, and HCDR3 comprising the amino acid sequences of SEQ ID NOs: 8 or 16, 9 or 17, and 10, respectively; and (ii) a first light chain variable region (VL1) comprising light chain complementarity determining region 1 (LCDR1), LCDR2, and LCDR3 comprising the amino acid sequences of SEQ ID NOs: 11, 12, and 13, respectively; and (b) the second antigen-binding region comprises (i) a second heavy chain variable region (VH2) comprising heavy chain complementarity determining region 1 (HCDR1), HCDR2, and HCDR3 comprising the amino acid sequences of SEQ ID NOs: 1, 2, and 3, respectively; and (ii) a second light chain variable region (VL2) comprising light chain complementarity determining region 1 (LCDR1), LCDR2, and LCDR3 comprising the amino acid sequences of SEQ ID NOs: 4, 5, and 6, respectively.

In certain embodiments, the VH1 comprises an amino acid sequence at least 90% identical to SEQ ID NO:14; and the VL1 comprises an amino acid sequence at least 90% identical to SEQ ID NO:15. In certain embodiments, the VH1 comprises the amino acid sequence of SEQ ID NO:14; and the VL1 comprises the amino acid sequence of SEQ ID NO:15.

In certain embodiments, the second antigen-binding region comprises a single chain fragment variable (scFv) antibody or antigen binding fragment thereof comprising the VH2 and VL2. The scFv can, for example, comprise an amino acid sequence at least 90% identical to SEQ ID NO:7. In certain embodiments, the scFV comprises the amino acid sequence of SEQ ID NO:7.

In certain embodiments, the multispecific antibody or antigen binding fragment thereof comprises two first antigen binding regions. The multispecific antibody or antigen binding fragment thereof of can, for example, comprise (i) a first heavy chain (HC1) comprising the VH1, a first heavy chain constant region comprising a first Fc region (Fc1), and the scFv; (ii) a second heavy chain (HC2) comprising the VH1 and a second heavy chain constant region comprising a second Fc region (Fc2); and (iii) a first and second light chain (LC) each comprising the VL1 and a light chain constant region.

In certain embodiments, the scFv is linked to the carboxy terminus of the first heavy chain constant region via a linker, more particularly a linker comprising the amino acid sequence of SEQ ID NO:27.

In certain embodiments, the Fc1 and Fc2 each comprise one or more heterodimeric mutations, such as a first and a second modified heterodimeric CH3 domains, respectively, as compared to a wild-type Fc region; particularly, the Fc1 comprises amino acid modifications at positions T350, L351, F405, and Y407, and the Fc2 comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat, more particularly, the Fc1 comprises amino acid modifications T350V, L351Y, F405A and Y407V, and the Fc2 comprises amino acid modifications T350V, T366L, K392L and T394W.

In certain embodiments, the Fc1 and Fc2 each comprise one or more heterodimeric mutations, such as a first and a second modified heterodimeric CH3 domains, respectively, as compared to a wild-type Fc region, wherein the Fc1 comprises amino acid modifications at positions T364, L366, and Y406, and the Fc2 comprises amino acid modifications at positions T365, wherein the amino acid modification at position T364 is T364S; the amino acid modification at position L366 is L366A; the amino acid modification at position Y406 is Y406V; the amino acid modification at position T365 is T365W, and wherein the numbering of amino acid residues is according to the EU index as set forth in Kabat, more particularly, the Fc1 comprises amino acid modifications T364S, L366A, and Y406V, and the Fc2 comprises amino acid modifications T365W.

In certain embodiments, at least one of the Fc1 and Fc2 comprises one or more mutations that enhance binding of the multispecific antibody or antigen binding fragment thereof to the neonatal Fc receptor (FcRn), preferably the one or more mutations enhance the binding at an acidic pH, more preferably the at least one of the Fc1 and Fc2 has the M252Y/S254T/T256E (YTE) mutations, wherein the numbering of amino acid residues is according to the EU index as set forth in Kabat.

In certain embodiments, at least one of the Fc1 and Fc2 comprises one or more mutations that reduce or eliminate the effector function, preferably the at least one of the Fc1 and Fc2 has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

Also provided is a multispecific antibody or antigen binding fragment thereof comprising a first heavy chain comprising an amino acid sequence at least 90% identical to SEQ ID NO:24, and a first light chain comprising an amino acid sequence at least 90% identical to SEQ ID NO:25, a second heavy chain comprising an amino acid sequence at least 90% identical to SEQ ID NO:26, and a second light chain comprising an amino acid sequence at least 90% identical to SEQ ID NO:25. In certain embodiments, the first heavy chain comprises the amino acid sequence of SEQ ID NO:24, the first light chain comprises the amino acid sequence of SEQ ID NO:25, the second heavy chain comprises the amino acid sequence of SEQ ID NO:26, and the second light chain comprises the amino acid sequence of SEQ ID NO:25.

Another general aspect of the application relates to an isolated nucleic acid encoding the multispecific antibody or antigen-binding fragment thereof of the application. Also provided is a vector comprising the isolated nucleic acid of the application, a host cell comprising the nucleic acid or the vector of the application.

Another general aspect of the application relates to a method of producing the multispecific antibody or antigen-binding fragment thereof of the application. The method comprises culturing a host cell of the application under conditions to produce the multispecific antibody or antigen-binding fragment thereof and recovering the multispecific antibody or antigen-binding fragment thereof from the cell or cell culture.

Further provided is a pharmaceutical composition comprising multispecific antibody or antigen binding fragment thereof of the application and a pharmaceutically acceptable carrier.

Another general aspect of the application relates to a method of treating or detecting a neurological disorder in a subject in need thereof, comprising administering to the subject an effective amount of the multispecific antibody or antigen binding fragment thereof or a pharmaceutical composition of the application. Preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

Also provided are methods of treating a condition associated with the formation of plaques containing beta-amyloid protein in a subject in need thereof. The methods comprise administering the multispecific antibody or antigen binding fragment thereof of the application or the pharmaceutical composition of the application to the subject in need thereof. The condition can, for example be Alzheimer's disease. The condition can, for example, be selected form the group consisting of dementia associated with Trisomy 21 (Down's Syndrome), diffuse Lewy body disease, inclusion body myositis, cerebral amyloid angiopathy and hereditary cerebral hemorrhage with amyloidosis of the Dutch-type (HCHWA-D).

Also provided are methods of reducing plaques associated with Alzheimer's disease in a subject in need thereof. The methods comprise administering the multispecific antibody or antigen binding fragment thereof of the application or the pharmaceutical composition of the application to the subject in need thereof.

Also provided are methods of preventing seeding activity of 3pE Aβ in a subject in need thereof. The methods comprise administering the multispecific antibody or antigen binding fragment thereof of the application or the pharmaceutical composition of the application to the subject in need thereof.

Other aspects, features and advantages of the invention will be apparent from the following disclosure, including the detailed description of the invention and its preferred embodiments and the appended claims.

### BRIEF DESCRIPTION OF THE FIGURES

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise embodiments shown in the drawings.

FIG. 1 is an illustration of a multispecific antibody or antigen binding fragment thereof of the application.

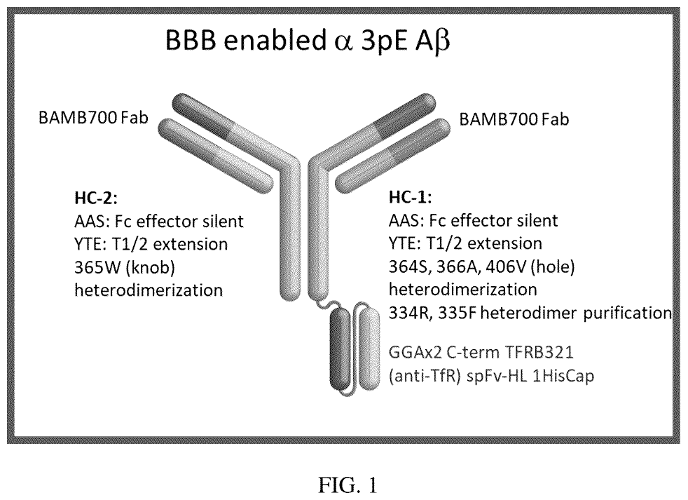

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N. Y., (1988).

Depending on the amino acid sequence of the constant domain of their heavy chains, full length antibodies can be assigned to different “classes”. There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

An “antibody” can also be a single variable domain on a heavy chain (VHH) antibody, also referred to as a heavy chain only antibody (HcAb), which are devoid of light chains and can be naturally produced by camelids or sharks. The antigen binding portion of the HcAb is comprised of a VHH fragment.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia, coli cells or Bacillus subtilis cells, etc.

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. Antigen-binding fragments can also be a single-domain antibody (sdAb), also known as a nanobody, which is an antibody fragment consisting of a single monomeric variable antibody domain (VHH). While some antigen-binding fragments of an antibody can be obtained by actual fragmentation of a larger antibody molecule (e.g., enzymatic cleavage), most are typically produced by recombinant techniques. The antibodies of the invention can be prepared as full-length antibodies or antigen-binding fragments thereof. Examples of antigen-binding fragments include Fab, Fab′, F(ab)₂, F(ab′)₂, F(ab)₃, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. In other embodiments, the antibody of choice is a single-chain Fv fragment (scFv). See WO 1993/16185; U.S. Pat. Nos. 5,571,894; and 5,587,458. The antibody fragment may also be a “linear antibody”, e.g., as described in U.S. Pat. No. 5,641,870, for example. Such linear antibody fragments can be monospecific or bispecific.

The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g., with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention can include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US Patent Application 20060073137, and U.S. Pat. Nos. 6,750,325, 6,632,927, 6,639,055, 6,548,640, 6,407,213, 6,180,370, 6,054,297, 5,929,212, 5,895,205, 5,886,152, 5,877,293, 5,869,619, 5,821,337, 5,821,123, 5,770,196, 5,777,085, 5,766,886, 5,714,350, 5,693,762, 5,693,761, 5,530,101, 5,585,089, and 5,225,539.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide can contain fewer or additional amino acids corresponding to a shortening of, or insertion into, a FR or CDR of the variable domain. For example, a heavy chain variable domain can include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence.

“Framework region” or “FR” residues are those VH or VL residues other than the CDRs as herein defined.

An “epitope” or “binding site” is an area or region on an antigen to which an antigen-binding peptide (such as an antibody) specifically binds. A protein epitope can comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide).

A “paratope” is an area or region of an antigen-binding portion of an antibody that specifically binds an antigen. Unless otherwise stated or clearly contradicted by context, a paratope can comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al, J. Chromatogr. B 848:79-87 (2007).

The term “administering” with respect to the methods of the invention, means a method for therapeutically or prophylactically preventing, treating or ameliorating a syndrome, disorder or disease as described herein by using a conjugate of the invention or a form, composition or medicament thereof. Such methods include administering an effective amount of said antibody, antigen-binding fragment thereof, or conjugate, or a form, composition or medicament thereof at different times during the course of a therapy or concurrently in a combination form. The methods of the invention are to be understood as embracing all known therapeutic treatment regimens.

The ability of a target antibody to “block” the binding of a target molecule to a natural target ligand, means that the antibody, in an assay using soluble or cell-surface associated target and ligand molecules, can detectably reduce the binding of a target molecule to the ligand in a dose-dependent fashion, where the target molecule detectably binds to the ligand in the absence of the antibody.

The “blood-brain barrier” or “BBB” refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A “blood-brain barrier receptor” (abbreviated “R/BBB” herein) is an extracellular membrane-linked receptor protein expressed on brain endothelial cells which is capable of transporting molecules across the BBB or be used to transport exogenous administrated molecules. Examples of R/BBB include, but are not limited to, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is transferrin receptor (TfR).

The “central nervous system” or “CNS” refers to the complex of nerve tissues that control bodily function and includes the brain and spinal cord.

A “conjugate” as used herein refer to a protein covalently linked to one or more heterologous molecule(s), including but not limited to a therapeutic peptide or protein, an antibody, a label, or a neurological disorder drug.

As used herein the term “coupled” refers to the joining or connection of two or more objects together. When referring to chemical or biological compounds, coupled can refer to a covalent connection between the two or more chemical or biological compounds. By way of a non-limiting example, an antibody of the invention can be coupled with a peptide of interest to form an antibody coupled peptide. An antibody coupled peptide can be formed through specific chemical reactions designed to conjugate the antibody to the peptide. In certain embodiments, an antibody of the invention can be covalently coupled with a peptide of the invention through a linker. The linker can, for example, be first covalently connected to the antibody or the peptide, then covalently connected to the peptide or the antibody.

An “effective amount” or “therapeutically effective amount” of an agent, e.g., a pharmaceutical formulation, refers to an amount effective, at dosages and for periods of time necessary, to achieve the desired therapeutic or prophylactic result.

A “linker” as used herein refers to a chemical linker or a single chain peptide linker that covalently connects two different entities. A linker can be used to connect any two of an antibody or a fragment thereof, a blood brain barrier shuttle, a fusion protein and a conjugate of the present invention. The linker can connect, for example, the VH and VL in scFv, or the antibody or antigen-binding fragment thereof with a therapeutic molecule, such as a second antibody. In some embodiments, if the monovalent binding entity comprises a scFv directed to TfR, preferably huTfR1, and the therapeutic molecule comprises an antibody directed to a CNS target, such as pyroglutamate amyloid-β, then the linker can connect the scFv to the antibody directed to pyroglutamate amyloid-β. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (including, but not limited to, Alzheimer disease and supranuclear palsy), prion diseases (including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

A “neurological disorder drug” is a drug or therapeutic agent useful in treating or ameliorating the effects of one or more neurological disorder(s). Neurological disorder drugs of the invention include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “pharmaceutically acceptable carrier or diluent” means any substance suitable for use in administering to an individual. For example, a pharmaceutically acceptable carrier can be a sterile aqueous solution, such as phosphate buffer saline (PBS) or water-for-injection.

As used herein, “pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

Pharmaceutically acceptable acidic/anionic salts for use in the invention include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid. Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

“Polypeptide” or “protein” means a molecule that comprises at least two amino acid residues linked by a peptide bond to form a polypeptide. Small polypeptides of less than 50 amino acids may be referred to as “peptides”.

The phrases “sequence identity” or “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g., 90%, 91%, 92%, 93%, 94%, 95%, 97%, 98%, 99%, or 100% identity over the full-length of the amino acid sequences) can be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

The term “substantially identical” in the context of two amino acid sequences means that the sequences, when optimally aligned, such as by the programs GAP or BESTFIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

“Specific binding” or “specifically binds” or “binds” refer to antibody binding to an antigen or an epitope within the antigen with greater affinity than for other antigens. Typically, the antibody binds to the antigen or the epitope within the antigen with a dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a K_{D} that is at least one hundred fold less than its K_{D} for binding to a non-specific antigen (e.g., BSA, casein). K_{D} is the equilibrium dissociation constant, a ratio of k_{off}/kₒₙ, between the antibody and its antigen. K_{D} and affinity are inversely related. The “on-rate” (kₒₙ) is a constant used to characterize how quickly the antibody binds to its target. The “off-rate” (k_{off}) is a constant used to characterize how quickly an antibody dissociates from its target. The dissociation constant K_{D} can be measured using standard procedures. For example, the K_{D} of an antibody can be determined by using surface plasmon resonance, such as by using a biosensor system, e.g., a Biacore® system, or by using bio-layer interferometry technology, such as an Octet RED96 system. The smaller the value of the K_{D} of an antibody, the higher affinity that the antibody binds to a target antigen. Antibodies that specifically bind to the antigen or the epitope within the antigen can, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

The term “subject” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. When the subject is human, they can also be referred to as a “patient”.

The term “transferrin receptor” or “TfR,” as used herein, refers to a cell surface receptor necessary for cellular iron uptake by the process of receptor-mediated endocytosis. carrier protein for transferrin. A TfR is involved in iron uptake in vertebrates and is regulated in response to intracellular iron concentration. It imports iron by internalizing the transferrin-iron complex through receptor-mediated endocytosis. Two transferrin receptors in humans, transferrin receptor 1 and transferrin receptor 2, have been characterized. Both these receptors are transmembrane glycoproteins. TfR1 is a high affinity ubiquitously expressed receptor. TfR2 binds to transferrin with a 25-30-fold lower affinity than TfR1. The expression of TfR2 is restricted to certain cell types and is unaffected by intracellular iron concentrations. In one embodiment, the TfR is a human TfR comprising the amino acid sequence as in Schneider et al. Nature 311: 675-678 (1984), for example. It can have a molecular weight of about 180,000 Dalton, having two subunits each of apparent molecular weight of about 90,000 Dalton. Preferably, the TfR is a human TfR1.

A “target antigen” or “brain target,” as used herein, refers to an antigen and/or molecule expressed in the CNS, including the brain, which can be targeted with an antibody or small molecule. Examples of such antigens and/or molecules include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In some embodiments, the target antigen is BACE1. In some embodiments, the target antigen is amyloid beta, in particular, pyroglutamate amyloid-β.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

Antibodies or immunoglobulins can be assigned to five major classes, namely IgA, IgD, IgE, IgG and IgM, depending on the heavy chain constant domain amino acid sequence. IgG is the most stable of the five types of immunoglobulins, having a serum half-life in humans of about 23 days. IgA and IgG are further sub-classified as the isotypes IgA₁, IgA₂, IgG₁, IgG₂, IgG3 and IgG4. Each of the four IgG subclasses has different biological functions known as effector functions. These effector functions are generally mediated through interaction with the Fc receptor (FcγR) and/or by binding C1q and fixing complement. Binding to FcγR can lead to antibody dependent cell mediated cytolysis or antibody-dependent cellular cytotoxicity (ADCC), whereas binding to complement factors can lead to complement mediated cell lysis or complement-dependent cytotoxicity (CDC). A multispecific antibody or antigen binding fragment thereof of the invention, or a therapeutic or diagnostic antibody to be conjugated or fused to the multispecific antibody or antigen binding fragment thereof can have no or minimal effector function, but retains its ability to bind FcRn, the binding of which can be a primary means by which antibodies have an extended in vivo half-life.

Binding of FcγR or complement (e.g., C1q) to an antibody is caused by defined protein-protein interactions at the so-called Fc part binding site. Such Fc part binding sites are known in the art. Such Fc part binding sites include, e.g., characterized by the amino acids L234, L235, D270, N297, E318, K320, K322, P331, and P329 (numbering according to EU index of Kabat). In some embodiment, a multispecific antibody or antigen binding fragment thereof of the invention, or a therapeutic or diagnostic antibody to be conjugated or fused to the multispecific antibody or antigen binding fragment thereof contains one or more substitutions in one or more Fc part binding sites to eliminate the effector function. For example, a multispecific antibody or antigen binding fragment thereof of the invention, or a therapeutic or diagnostic antibody to be conjugated or fused to the multispecific antibody or antigen binding fragment thereof can contain a Fc region containing one or more of the following substitutions: substitution of proline for glutamate at residue 233, alanine or valine for phenylalanine at residue 234 and alanine or glutamate for leucine at residue 235 (EU numbering, Kabat, E. A. et al. (1991) Sequences of Proteins of Immunological Interest, 5th Ed. U.S. Dept. of Health and Human Services, Bethesda, Md., NIH Publication no. 91-3242). Preferably, the antibody of interest contains one, two or three mutations of L234A, L235A and P331S (EU numbering, Kabat).

Antibodies of subclass IgG1, IgG2, and IgG3 usually show complement activation including C1q and C3 binding, whereas IgG4 does not activate the complement system and does not bind C1q and/or C3. Human IgG4 Fc region has reduced ability to bind FcγR and complement factors compared to other IgG sub-types. Preferably, a multispecific antibody or antigen binding fragment thereof of the invention, or a therapeutic or diagnostic antibody to be conjugated or fused to the multispecific antibody or antigen binding fragment thereof comprises a Fc region derived from human IgG4 Fc region. More preferably, the Fc region contains human IgG4 Fc region having substitutions that eliminate effector function. For example, removing the N-linked glycosylation site in the IgG4 Fc region by substituting Ala for Asn at residue 297 (EU numbering) is another way to ensure that residual effector activity is eliminated.

### Multispecific Anti-Pyroglutamate Amyloid-$/Anti-Transferrin Receptor (TfR) Antibodies and Antigen Binding Fragments Thereof

### Anti-Pyroglutamate Amyloid-β Binding Regions/Anti-Pyroglutamate Amyloid-β Antibodies and Antigen Binding Fragments Thereof

Anti-pyroglutamate amyloid-β antibodies and antigen binding fragments thereof were previously disclosed in WO2020/193644, which is incorporated by reference herein in its entirety. In one general aspect, the application relates to a multispecific antibody or antigen binding fragment thereof comprising an antigen-binding region based on the previously described anti-pyroglutamate amyloid-β antibodies and antigen binding fragments thereof.

As used herein, “pyroglutamate amyloid-β,” “AβpE3,” or “3pE Aβ” refers to a modified Aβ peptide that oligomerizes with Aβ42 and is deposited in the Alzheimer's disease (AD) brain. Pyroglutamate amyloid-β can act as a seed for misfolding Ab as a primary step in AD. Pyroglutamate amyloid-β is known in the art, see, e.g., Perez-Garmendia et al., Curr. Neuropharma. 11(5):491-8 (2013); Wittnam et al., JBC 287(11):8154-62 (2012); and Wang et al., Alzheimer's Dement 12:e12029 (2020).

### Anti-TfR Antigen Binding Regions/Anti-TfR Antibodies and Antigen Binding Fragments Thereof

Anti-TfR antibodies and antigen binding fragments thereof were previously disclosed in WO2021/0205358, which is incorporated by reference herein in its entirety. In one general aspect, the application relates to a multispecific antibody or antigen binding fragment thereof comprising an antigen-binding region based on the previously described anti-TfR antibodies and antigen binding fragments thereof. The anti-TfR antigen-binding regions are capable of binding to a primate TfR, such as a human TfR or a monkey TfR, and the antigen-binding region can be optimized for delivering an agent to the brain of a subject in need thereof. The relationship between the binding affinity of an anti-TfR antibody to the TfR and transcytosis efficiency has been described previously as improved transcytosis with decreased affinity for TfR (Yu, Zhang et al. 2011, Sci Transl Med 3(84): 84ra44) In WO2021/0205358, which is incorporated by reference herein in its entirety, a more nuanced relationship between affinity and transcytosis efficiency than what has been previously described was surprisingly discovered, with influence from both on- and off-rates impacting brain concentration. In particular, a neutral off-rate that is neither too fast nor too slow is required for optimal brain PK and PD of an agent (such as an mAb) to be efficiently delivered by the anti-TfR antibody or antigen binding fragment thereof.

Preferably, an anti-TfR antigen-binding region of the application is pH-sensitive, e.g., it has different binding affinities to TfR at different pHs. For example, an anti-TfR antigen-binding region of the application can bind to cell surface TfR at a neutral pH, such as physiological pH (e.g., pH 7.4), with high affinity, but upon internalization into an endosomal compartment, dissociates from TfR at an acidic pH, such as the relatively lower pH (pH 5.0-6.0). Affinity is a measure of the strength of binding between two moieties, e.g., an antibody and an antigen. Affinity can be expressed in several ways. One way is in terms of the dissociation constant (K_{D}) of the interaction. K_{D} can be measured by routine methods, include equilibrium dialysis or by directly measuring the rates of antigen-antibody dissociation and association, the k_{off} (kd or k_{dis}) and kₒₙ(or ka) rates, respectively (see e.g., Nature, 1993 361:186-87). The ratio of k_{off}/kₒₙ cancels all parameters not related to affinity and is equal to the dissociation constant K_{D} (see, generally, Davies et al., Annual Rev Biochem, 1990 59:439-473). Thus, a smaller K_{D} means a higher affinity. Another expression of affinity is Kₐ, which is the inverse of K_{D}, or kₒₙ/k_{off}. Thus, a higher Kₐ means a higher affinity. For example, an anti-TfR antigen-binding region for use in a composition and/or method of the application can be an anti-TfR antigen-binding region that binds to a TfR with a K_{D} of 1 nanomolar (nM, 10⁻⁹ M) or more at a neutral pH (e.g., pH 6.8-7.8), such as a physiological pH (e.g., pH 7.4), and dissociates from TfR with a k_{dis} of 10⁻⁴ sec⁻¹ or more at an acidic pH (e.g., pH 4.5-6.0), such as pH5.0).

Accordingly, a general aspect of the application relates to an anti-TfR antigen-binding region for delivering an agent to the brain of a subject in need thereof, wherein the anti-TfR antigen-binding region binds to a transferrin receptor (TfR), preferably human TfR1, with a dissociation constant K_{D} of at least 1 nM, preferably 1 nM to 500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

In one embodiment, the anti-TfR antigen-binding region of the application has an off-rate constant k_{d} of 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between, at the neutral pH.

### Multispecific Antibodies and Antigen Binding Fragments Thereof

In certain embodiments, the multispecific antibody or antigen binding fragment thereof comprises at least one first antigen-binding region capable of binding specifically to pyroglutamate amyloid-β and a second antigen-binding region capable of binding specifically to transferrin receptor (TfR). In certain embodiments, (a) the first antigen-binding region comprises (i) a first heavy chain variable region (VH1) comprising heavy chain complementarity determining region 1 (HCDR1), HCDR2, and HCDR3 comprising the amino acid sequences of SEQ ID NOs: 8 or 16, 9 or 17, and 10, respectively; and (ii) a first light chain variable region (VL1) comprising light chain complementarity determining region 1 (LCDR1), LCDR2, and LCDR3 comprising the amino acid sequences of SEQ ID NOs: 11, 12, and 13, respectively; and (b) the second antigen-binding region comprises (i) a second heavy chain variable region (VH2) comprising heavy chain complementarity determining region 1 (HCDR1), HCDR2, and HCDR3 comprising the amino acid sequences of SEQ ID NOs: 1, 2, and 3, respectively; and (ii) a second light chain variable region (VL2) comprising light chain complementarity determining region 1 (LCDR1), LCDR2, and LCDR3 comprising the amino acid sequences of SEQ ID NOs: 4, 5, and 6, respectively.

In certain embodiments, the VH1 comprises an amino acid sequence at least 90% identical to SEQ ID NO:14; and the VL1 comprises an amino acid sequence at least 90% identical to SEQ ID NO:15. In certain embodiments, the VH1 comprises the amino acid sequence of SEQ ID NO:14; and the VL1 comprises the amino acid sequence of SEQ ID NO:15.

In certain embodiments, the second antigen-binding region comprises a single chain fragment variable (scFv) antibody or antigen binding fragment thereof comprising the VH2 and VL2. The scFv can, for example, comprise an amino acid sequence at least 90% identical to SEQ ID NO:7. In certain embodiments, the scFV comprises the amino acid sequence of SEQ ID NO:7.

In certain embodiments, the multispecific antibody or antigen binding fragment thereof comprises two first antigen binding regions. The multispecific antibody or antigen binding fragment thereof of can, for example, comprise (i) a first heavy chain (HC1) comprising the VH1, a first heavy chain constant region comprising a first Fc region (Fc1), and the scFv; (ii) a second heavy chain (HC2) comprising the VH1 and a second heavy chain constant region comprising a second Fc region (Fc2); and (iii) a first and second light chain (LC) each comprising the VL1 and a light chain constant region.

Preferably, the anti-TfR antigen-binding region is single-chain variable fragment (scFv) comprising the heavy chain variable region (Hv) covalently linked to the light chain variable region (Lv) via a flexible linker. The scFv can retain the specificity of the original immunoglobulin, despite removal of the constant regions and the introduction of the linker. In a scFv, the order of the domains can be either Hv-linker-Lv, or Lv-linker-Hv. The linker can be designed de novo or derived from known protein structure to provide a compatible length and conformational in bridging the variable domains of a scFv without serious steric interference. The linker can have 10 to about 25 amino acids in length. Preferably, the linker is a peptide linker spanning about 3.5 nm (35 Å) between the carboxy terminus of the variable domain and the amino terminus of the other domain without affecting the ability of the domains to fold and form an intact antigen-binding site (Huston et al., Methods in Enzymology, vol. 203, pp. 46-88, 1991, which is incorporated herein by reference in its entirety). The linker preferably comprises a hydrophilic sequence in order to avoid intercalation of the peptide within or between the variable domains throughout the protein folding (Argos, Journal of Molecular Biology, vol. 211, no. 4, pp. 943-958, 1990). For example, the linker can comprise Gly and Ser residues and/or together with the charged residues such as Glu, Thr and Lys interspersed to enhance the solubility. In one embodiment, the linker has the amino acid sequence of SEQ ID NO: 27 (GGGSGGSGGCPPCGGSGG). Any other suitable linker can also be used in view of the present disclosure. In certain embodiments, the scFv is linked to the carboxy terminus of the first heavy chain constant region via a linker, more particularly a linker comprising the amino acid sequence of SEQ ID NO:27.

Also provided is a multispecific antibody or antigen binding fragment thereof comprising a first heavy chain comprising an amino acid sequence at least 90% identical to SEQ ID NO:24, and a first light chain comprising an amino acid sequence at least 90% identical to SEQ ID NO:25, a second heavy chain comprising an amino acid sequence at least 90% identical to SEQ ID NO:26, and a second light chain comprising an amino acid sequence at least 90% identical to SEQ ID NO:25. In certain embodiments, the first heavy chain comprises the amino acid sequence of SEQ ID NO:24, the first light chain comprises the amino acid sequence of SEQ ID NO:25, the second heavy chain comprises the amino acid sequence of SEQ ID NO:26, and the second light chain comprises the amino acid sequence of SEQ ID NO:25.

According to another particular aspect, the invention relates to an isolated multispecific antibody or antigen-binding fragment thereof of the invention, wherein the multispecific antibody or antigen-binding fragment thereof is chimeric.

According to another particular aspect, the invention relates to an isolated multispecific antibody or antigen-binding fragment thereof of the invention, wherein the multispecific antibody or antigen-binding fragment thereof is human or humanized.

In another general aspect, the invention relates to an isolated nucleic acid encoding a the multispecific antibody or antigen-binding fragment thereof of the invention. It will be appreciated by those skilled in the art that the coding sequence of a protein can be changed (e.g., replaced, deleted, inserted, etc.) without changing the amino acid sequence of the protein.

Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding the multispecific antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated nucleic acid encoding the multispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

### Brain Shuttle Construct

An optimized RMT brain delivery platform is developed using the transferrin receptor (TfR) by enhancing the intrinsic transcytosis efficiency, extending peripheral pharmacokinetics, and engineering for an acceptable safety profile while maintaining efficacy of the therapeutic mAb. The interplay between transcytosis receptor affinity and brain concentration in human TfR knock-in mice is studied. A thorough study of binding kinetics demonstrate that for optimal brain PK and PD of mAbs, a neutral off-rate that is neither too fast nor too slow is required. The enhanced brain delivery observed in mice was confirmed in cynomolgus monkey.

It is also discovered that engineered antibody constant region with increased binding to the neonatal Fc receptor (FcRn) resulted in decreased peripheral clearance and enhancement in brain concentration.

Additional Fc mutations are introduced to abolish binding to Fc gamma receptors (FcγR) and avoid effector function mediated toxicity. When coupled with a high affinity anti-pyroglutamate amyloid-β binding mAb, these mutations prevent effector function mediated toxicity in the periphery while maintaining antibody dependent phagocytosis (ADP) through a novel, non-FcγR mechanism for microglial uptake and target degradation. This mechanism is dependent upon internalization through the TfR receptor and is more efficient in promoting target degradation than traditional FcγR mediated ADP without the stimulating the secretion of pro-inflammatory cytokines.

In certain embodiments, the Fc1 and Fc2 each comprise one or more heterodimeric mutations, such as a first and a second modified heterodimeric CH3 domains, respectively, as compared to a wild-type Fc region; particularly, the Fc1 comprises amino acid modifications at positions T350, L351, F405, and Y407, and the Fc2 comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat, more particularly, the Fc1 comprises amino acid modifications T350V, L351Y, F405A and Y407V, and the Fc2 comprises amino acid modifications T350V, T366L, K392L and T394W.

In certain embodiments, the Fc1 and Fc2 each comprise one or more heterodimeric mutations, such as a first and a second modified heterodimeric CH3 domains, respectively, as compared to a wild-type Fc region, wherein the Fc1 comprises amino acid modifications at positions T364, L366, and Y406, and the Fc2 comprises amino acid modifications at positions T365, wherein the amino acid modification at position T364 is T364S; the amino acid modification at position L366 is L366A; the amino acid modification at position Y406 is Y406V; the amino acid modification at position T365 is T365W, and wherein the numbering of amino acid residues is according to the EU index as set forth in Kabat, more particularly, the Fc1 comprises amino acid modifications T364S, L366A, and Y406V, and the Fc2 comprises amino acid modifications T365W

In certain embodiments, at least one of the Fc1 and Fc2 comprises one or more mutations that enhance binding of the multispecific antibody or antigen binding fragment thereof to the neonatal Fc receptor (FcRn), preferably the one or more mutations enhance the binding at an acidic pH, more preferably the at least one of the Fc1 and Fc2 has the M252Y/S254T/T256E (YTE) mutations, wherein the numbering of amino acid residues is according to the EU index as set forth in Kabat.

In certain embodiments, at least one of the Fc1 and Fc2 comprises one or more mutations that reduce or eliminate the effector function, preferably the at least one of the Fc1 and Fc2 has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

Accordingly, in one general aspect, the application relates to an antibody-targeted brain delivery system comprising an anti-TfR antigen binding-region of the application. The anti-TfR antigen binding-region can be used to deliver a therapeutic or diagnostic agent into a cell (e.g., a cancer cell) or a BBB system. Agents that can be delivered include any neurological disorder drug or agent that can be used to detect or analyze a neurological disorder drug. For example, such agent can be neurotrophic factors, including, but not limited to, nerve growth factor (NGF), brain derived neurotrophic factor (BDNF), ciliary neurotrophic factor (CNTF), glial cell-line neurotrophic factor (GDNF) and insulin-like growth factor (IGF); neuropeptides, including, but not limited to, Substance P, neuropeptideY, vasoactive intestinal peptide (VIP), gamma-amino-butyric acid (GABA), dopamine, cholecystokinin (CCK), endorphins, enkephalins and thyrotropin releasing hormone (TRH); cytokines; anxiolytic agents; anticonvulsants; polynucleotides and transgenes, including, for example, small interfering RNAs and/or antisense oligos; or antibodies or antigen binding fragments thereof that bind to a brain target. An anti-hTfR antigen binding-region of the application can be an effective means to enhance the delivery of an agent of interest from the blood into the brain and function there.

In particular, an agent of interest can be delivered in a combined form or linked to an anti-TfR antigen binding-region of the application, parenterally, e.g., intravenously. For example, the agent can be non-covalently attached to the anti-TfR antigen binding-region. The agent can also be covalently attached to the anti-TfR antigen binding-region to form a conjugate. In certain embodiments, the conjugation is by construction of a protein fusion (i.e., by genetic fusion of the two genes encoding an anti-TfR antigen binding-region and a neurological disorder drug and expression as a single protein). Known methods can be used to link an agent to an antibody or antigen binding fragment thereof in view of the present disclosure. See, for example, Wu et al., Nat Biotechnol., 23(9):1137-46, 2005; Trail et al., Cancer Immunol Immunother., 52(5):328-37, 2003; Saito et al., Adv Drug Deliv Rev., 55(2):199-215, 2003; Jones et al., Pharmaceutical Research, 24(9):1759-1771, 2007.

In some embodiments, a therapeutic or diagnostic agent to be delivered to the brain and an anti-TfR antigen binding-region can be covalently linked together (or conjugated) via a non-peptide linker or a peptide linker. Examples of non-peptide linkers include, but are not limited to, polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof. A peptide linker can be a peptide chain consisting of 1 to 50 amino acids linked by peptide bonds or a derivative thereof, whose N-terminus and C-terminus can be covalently linked to an anti-TfR antigen binding-region.

In certain embodiments, a conjugate of the application is a multispecific antibody or antigen binding fragment thereof comprising a first antigen binding region which binds pyroglutamate amyloid-β and a second antigen binding region which binds TfR. Techniques for making multi-specific antibodies include, but are not limited to, recombinant co-expression of two immunoglobulin heavy chain-light chain pairs having different specificities (see Milstein and Cuello, Nature 305: 537, 1983), WO 93/08829, and Traunecker et al, EMBO J. 10: 3655, 1991), and “knob-in-hole” engineering (see, e.g., U.S. Pat. No. 5,731,168). Multi-specific antibodies can also be made by engineering electrostatic steering effects (WO 2009/089004A1); cross-linking two or more antibodies or fragments (see, e.g., U.S. Pat. No. 4,676,980, and Brennan et al, Science, 229: 81, 1985); using leucine zippers (see, e.g., Kostelny et al, J. Immunol., 148(5): 1547-1553, 1992)); using “diabody” technology (see, e.g., Hollinger et al, Proc. Natl. Acad. Sci. USA, 90:6444-6448, 1993)); using single-chain Fv (sFv) dimers (see, e.g. Gruber et al, J. Immunol, 152:5368 (1994)); and preparing trispecific antibodies as described, e.g., in Tutt et al. J. Immunol. 147: 60, 1991. A multispecific antibody of the application also encompasses antibodies having three or more functional antigen binding sites, including “Octopus antibodies” or “dual-variable domain immunoglobulins” (DVDs) (see, e.g. US 2006/0025576A1, and Wu et al. Nature Biotechnology, 25(11):1290-7, 2007). A multispecific antibody of the application also encompasses a “Dual Acting Fab” or “DAF” comprising an antigen binding region that binds to TfR as well as pyroglutamate amyloid-β(see, US 2008/0069820, for example). In one embodiment, the antibody is an antibody fragment, various such fragments being disclosed herein.

In one embodiment, a multispecific antibody of the application is a fusion construct comprising an anti-TfR antigen binding-region of the application covalently linked (or fused) to a second antibody or antigen binding fragment thereof. Preferably, the second antibody or antigen binding fragment thereof binds to a brain target, such as pyroglutamate amyloid-β, as described herein. The anti-TfR antigen binding-region can be fused to the carboxy- and/or amino-terminus of a light and/or heavy chain of the second antibody or antigen binding fragment thereof, directly or via a linker.

In one embodiment, the anti-TfR antigen binding-region is fused to the carboxy-terminus of a light chain of the second antibody or antigen binding fragment thereof, directly or via a linker.

In another embodiment, the anti-TfR antigen binding-region is fused to the amino-terminus of a light chain of the second antibody or antigen binding fragment thereof, directly or via a linker.

In another embodiment, the anti-TfR antigen binding-region is fused to the carboxy-terminus of a heavy chain of the second antibody or antigen binding fragment thereof, directly or via a linker.

In another embodiment, the anti-TfR antigen binding-region is fused to the amino-terminus of a heavy chain of the second antibody or antigen binding fragment thereof, directly or via a linker.

In a preferred embodiment, a fusion construct of the application comprises an anti-TfR antigen binding-region, preferably an anti-huTfR1 VHH or scFv fragment, of the application covalently linked, via a linker, to the carboxy terminus of only one of the two heavy chains of a second antibody or antigen binding fragment thereof that binds to pyroglutamate amyloid-β. Preferably, the linker has the amino acid sequence of SEQ ID NO: 27.

To facilitate the formation of a heterodimer between the two heavy chains, e.g., one with a fusion of the anti-TfR antigen binding-region and one without, or one containing the Fc for the anti-TfR arm and one for the anti-pyroglutamate amyloid-β arm, heterodimeric mutations introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the invention. In some embodiment, a modified CH3 as described herein is used to facilitate the formation of a heterodimer between the two heavy chains.

In addition to the heterodimeric mutations, other mutations can also be introduced. In some embodiments, the Fc region of the fusion construct or bispecific antibody further comprises one or more mutations that alter (increase or diminish), preferably eliminate ADCC/CDC (such as the AAS mutations described herein), and/or one or more mutations that alter (increase or diminish), preferably increase, the binding of the fusion construct or bispecific antibody to FcRn (such as the YTE mutations described herein). In some embodiment, one or more cysteine residues in the fusion construct or bispecific antibody are substituted with other amino acids, such as serine.

A conjugate, such as a multispecific antibody or fusion construct, of the application can be produced by any of a number of techniques known in the art in view of the present disclosure. For example, it can be expressed from a recombinant host cells, wherein expression vector(s) encoding the heavy and light chains of the fusion construct or multi-specific antibody is (are) transfected into a host cell by standard techniques. The host cells can be prokaryotic or eukaryotic host cells.

In an exemplary system, one or more recombinant expression vectors encoding the heterodimeric two heavy chains and the light chains of a fusion construct of the application is/are introduced into host cells by transfection or electroporation. The selected transformant host cells are cultured to allow for expression of the heavy and light chains under conditions sufficient to produce the fusion construct, and the fusion construct is recovered from the culture medium.

Standard molecular biology techniques are used to prepare the recombinant expression vector, transfect the host cells, select for transformants, culture the host cells and recover the protein construct from the culture medium.

### Pharmaceutical Composition and Related Methods

The invention also relates to pharmaceutical compositions, methods of preparation and methods for use thereof.

In another general aspect, the invention relates to a pharmaceutical composition, comprising a multispecific antibody or antigen binding fragment thereof of the invention and a pharmaceutically acceptable carrier. The multispecific antibody or antigen binding fragment thereof of the invention is also useful in the manufacture of a medicament for therapeutic applications mentioned herein. The pharmaceutically acceptable carrier can be any suitable excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application.

Accordingly, in one embodiment, the application relates to a method of transporting a therapeutic or diagnostic agent across the blood-brain barrier (BBB) comprising exposing a multispecific antibody or antigen binding fragment thereof of the invention coupled to the therapeutic or diagnostic agent to the blood-brain barrier such that the multispecific antibody or antigen binding fragment thereof transports the agent coupled thereto across the blood-brain barrier. In one embodiment, the agent is a neurological disorder drug. In another embodiment, the agent is an imaging agent or an agent for detecting a neurological disorder. Preferably, the multispecific antibody or antigen binding fragment thereof does not impair the binding of the TfR to its native ligand transferrin. The antibody specifically binds to TfR in such a manner that it does not inhibit binding of the TfR to transferrin. In some embodiment, the BBB is in a mammal, preferably a primate, such as a human, more preferably a human having a neurological disorder. In one embodiment, the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

In one embodiment, the multispecific antibody or antigen binding fragment thereof of the application, is used to detect a neurological disorder before the onset of symptoms and/or to assess the severity or duration of the disease or disorder. The multispecific antibody or antigen binding fragment thereof permits detection and/or imaging of the neurological disorder, including imaging by radiography, tomography, or magnetic resonance imaging (MRI).

In another embodiment, the multispecific antibody or antigen binding fragment thereof is used in treating a neurological disorder (e.g., Alzheimer's disease), comprising administering to a subject in need of the treatment an effective amount of the multispecific antibody or antigen binding fragment thereof. In some embodiments, the method further comprises administering to the subject an effective amount of at least one additional therapeutic agent.

In another embodiment, the invention relates to a method for preventing, ameliorating, treating and/or decreasing amyloid-beta deposition in amyloid-beta related conditions comprising administration of the multispecific antibodies or antigen binding fragments thereof as disclosed herein in a therapeutically effective amount to a subject in need thereof. Additional aspects of the invention include a pharmaceutical composition for preventing, ameliorating, treating and/or decreasing amyloid deposition in amyloid-beta related conditions comprising the antibodies or antigen binding fragments thereof as disclosed herein. Methods of the present invention comprise administering an effective amount of one or more antibodies or antigen binding fragments thereof described herein to a subject in need thereof.

In one aspect, the invention is directed to methods of preventing, ameliorating, treating and/or decreasing amyloid-beta deposition in conditions characterized by the formation of plaques containing beta-amyloid protein in humans, which method comprises administering, preferably peripherally, to a human in need of such treatment a therapeutically or prophylactically effective amount of a multispecific antibody according to the invention or an immunologically reactive fragment thereof, which multispecific antibody specifically binds to human Aβ3pE. In another aspect, the invention is directed to methods of inhibiting the formation of amyloid plaques and/or methods of clearing amyloid plaques in humans, which method comprises administering to a human subject in need of such inhibition or clearing an effective amount of a multispecific antibody according to the invention, wherein the multispecific antibody sequesters Aβ3pE peptide in the brain and induces altered Aβ3pE clearance in brain.

A subject in need thereof is a human suffering or predisposed to suffer from a condition characterized by the formation of plaques containing beta-amyloid protein. In one embodiment, the condition in Alzheimer's disease. In other embodiments, the condition is dementia associated with Trisomy 21 (Down's Syndrome), diffuse Lewy body disease, inclusion body myositis, cerebral amyloid angiopathy or hereditary cerebral hemorrhage with amyloidosis of the Dutch-type (HCHWA-D).

In an embodiment of the invention, multispecific antibodies or antigen binding fragments thereof of the invention bind to 3pE Aβ in plaque deposits. By binding to 3pE Aβ in plaque deposits, the multispecific antibody or antigen binding fragment thereof can induce plaque removal. Induction of plaque removal can be by activation of microglia around plaques and by destabilizing plaques by removing a stable Aβ form. Moreover, antibodies or antigen binding fragments thereof of the invention can prevent plaque seeding activity of 3pE A3. The possible enrichment of 3pE Aβ in plaque compared to vascular amyloid can increase the therapeutic safety window for immunotherapy.

In another embodiment, the application relates to the use of the multispecific antibody or antigen binding fragment thereof of the application in the manufacture or preparation of a medicament. In one embodiment, the medicament is for treatment of neurological disease or disorder. In a further embodiment, the medicament is for use in a method of treating neurological disease or disorder comprising administering to an individual having neurological disease or disorder an effective amount of the medicament.

Another general aspect of the application relates to a method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the multispecific antibody or antigen binding fragment thereof of the application, wherein the therapeutic antibody or antigen binding fragment thereof does not have effector function. For example, the therapeutic antibody or antigen binding fragment thereof can comprise one or more amino acid modifications that reduces or eliminates the effector function, such as the ADCC or CDC, such as mutations that reduce or abolish the binding to Fc gamma receptor. Such mutations can be at positions L234, L235, D270, N297, E318, K320, K322, P331, and P329, such as one, two or three mutations of L234A, L235A and P331S, wherein the numbering of amino acid residues is according to the EU index as set forth in Kabat. In one embodiment, the therapeutic antibody or antigen binding fragment thereof binds specifically to tau aggregates.

In some embodiments, the method further comprises administering to the subject an effective amount of at least one additional therapeutic agent. In certain embodiments, an additional therapeutic agent is a therapeutic agent effective to treat the same or a different neurological disorder as the multispecific antibody or antigen binding fragment thereof is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, 7-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug. The additional therapeutic agent can be administered in the same or separate formulations and administered together or separately with the multispecific antibody or antigen binding fragment thereof. The multispecific antibody or antigen binding fragment thereof of the application can be administered prior to, simultaneously with, and/or following, the administration of the additional therapeutic agent and/or adjuvant. The multispecific antibody or antigen binding fragment thereof of the application can also be used in combination with other interventional therapies such as, but not limited to, radiation therapy, behavioral therapy, or other therapies known in the art and appropriate for the neurological disorder to be treated or prevented.

The multispecific antibody or antigen binding fragment thereof of the application (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration, depending in part on whether the administration is brief or chronic. Various dosing schedules including but not limited to single or multiple administrations over various time-points, bolus administration, and pulse infusion are contemplated herein.

For the prevention or treatment of a disease, the appropriate dosage of the multispecific antibody or antigen binding fragment thereof of the application (when used alone or in combination with one or more other additional therapeutic agents) will depend on various factors, such as the type of disease to be treated, the type of antibody or conjugate, the severity and course of the disease, whether the multispecific antibody or antigen binding fragment thereof is administered for preventive or therapeutic purposes, previous therapy, the patient's clinical history and response to the antibody, the physiological state of the subject (including, e.g., age, body weight, health), and the discretion of the attending physician. Treatment dosages are optimally titrated to optimize safety and efficacy. The multispecific antibody or antigen binding fragment thereof is suitably administered to the patient at one time or over a series of treatments.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In another aspect, the application relates to an article of manufacture (such as a kit) containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a multispecific antibody or antigen binding fragment thereof of the application. The label or package insert indicates that the composition is used for treating the condition of choice. Moreover, the article of manufacture can include (a) a first container with a composition contained therein, wherein the composition comprises a multispecific antibody or antigen binding fragment thereof of the application; and (b) a second container with a composition contained therein, wherein the composition comprises a further cytotoxic or otherwise therapeutic agent. The article of manufacture in this embodiment of the invention can further include a package insert indicating that the compositions can be used to treat a particular condition. Optionally, the article of manufacture can further comprise a second (or third) container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

### In Vitro Methods

It is to be understood that all manner of immunoassays employing multispecific antibodies or antigen binding fragments thereof are contemplated for use in accordance with the presently preferred embodiments, including assays in which multispecific antibodies or antigen binding fragments thereof are bound to solid phases and assays in which antibodies are in liquid media. Methods of immunoassays that can be used to detect analytes using multispecific antibodies embodying features of the present invention include, but are not limited to, competitive (reagent limited) assays wherein labeled analyte (analyte analog) and analyte in a sample compete for antibodies and single-site immunometric assays wherein the multispecific antibody is labeled; and the like.

The multispecific antibodies or antigen binding fragments thereof according to the invention can be used in conventional immunological techniques for the detection of Aβ3pE or TfR wherever the peptides can occur, including biological samples and conditioned media from cell culture. Suitable immunological techniques are well known to those skilled in the art and include, for example, ELISA, Western Blot analysis, competitive or sandwich immunoassays and the like, and as is otherwise well known, they all depend on the formation of an antigen-antibody immune complex wherein for the purpose of the assay, the antibody or antigen binding fragment thereof can be detectably labelled with, e.g. radio, enzyme, luminescent or fluorescent labels or it can be immobilized on insoluble carriers. It is thus an object of the invention to provide immunoassays for the determination or detection of Aβ3pE and/or TfR or fragments thereof in a sample, the method comprising contacting the sample with an antibody or antigen binding fragment thereof to Aβ3pE and/or TfR or a fragment thereof according to the invention and determining whether an immune complex is formed between the antibody or antigen binding fragment thereof and the Aβ3pE or fragment thereof. These methods can either be performed on tissue samples or body fluid samples and generally comprise obtaining a sample from the body of a subject; contacting said sample with an imaging effective amount of a detectably labeled antibody or antigen binding fragment thereof according to the invention; and detecting the label to establish the presence of Aβ3pE and/or TfR or fragments thereof in the sample. The measuring methods using the antibodies or antigen binding fragments thereof of the present invention are not particularly limited. Any measuring method can be used as long as the amount of antibodies, antigens, or the antigen-antibody complexes corresponding to the amount of the antigens, in particular the amount of Aβ3pE and/or TfR or fragments thereof in solutions to be measured is detected by chemical or physical means, and calculated from standard curves prepared by the use of standard solutions containing the antigens in known amounts. For example, nephelometry, competitive methods, immunometric methods and sandwich methods are suitably used. With respect to sensitivity and specificity, it is particularly preferred to use sandwich methods.

In the sandwich methods, the test solutions are reacted with an insolubilized antibody, such as insolubilized multispecific antibody or the invention (the first reaction), further, the labeled secondary antibodies are reacted (the second reaction); the activity of the labeling agents on the insolubilized carriers is then assayed, whereby the amount of the Aβ3pE and/or TfR or fragments thereof in the test solutions can be determined. The first reaction and the second reaction can be conducted simultaneously or sequentially.

In measuring methods, labelling substances, radioisotopes, enzymes, fluorescent substances, luminous substances, etc. are used as labelling agents. Examples of the radioisotopes include ¹²⁵1, ¹³¹I, ³H and ¹⁴C. Enzymes are usually made detectable by conjugation of an appropriate substrate that, in turn catalyzes a detectable reaction. Examples thereof include, for example, beta-galactosidase, beta-glucosidase, alkaline phosphatase, peroxidase and malate dehydrogenase, preferably horseradish peroxidase. The luminous substances include, for example, luminol, luminol derivatives, luciferin, aequorin and luciferase. Further, the avidin-biotin systems can also be used for labelling the antibodies and immunogens of the present invention. When the immunogens or antibodies are insolubilized, either physical adsorption or chemical binding usually used for insolubilization or fixation of proteins or enzymes can be employed. Examples of the carriers include insoluble polysaccharides such as agarose, dextran, and cellulose, synthetic resins such as polystyrene, polyacrylamide and silicone polymers, and glass.

In a further embodiment for detecting or diagnosing β-amyloid-related diseases and/or other neurological diseases or conditions, a biological sample including tissue, body fluids, such as cerebrospinal fluid (CSF), blood, plasma, serum, urine, and the like, is contained and contacted with a suitable amount of a first antibody to produce an immune complex. The contact typically involves adding the sample to a solid matrix coated with the first antibody. The complex which results from contacting the sample with the first antibody is separated from the sample by elution. However, other methods of recovery can be employed. The recovered complex is contacted with at least one second antibody directed to an antigenic determinant on the antigen and capable of binding the antigen in the complex. The antigenic determinant to which the second antibody is directed can be the same one as to which the first antibody is directed due to the multiepitopic nature of the antigenic entity. Either the first or the second antibody can be made detectable using any of the labels described above. In a preferred embodiment, the second antibody is made detectable. The presence of the detectable antibody bound to the complex consisting of antigen bound to the first and second antibody can be readily detected using art-known techniques. By comparing the results obtained in the biological sample with those obtained on a control sample, the presence of or levels of altered Aβ3pE and/or TfR or fragments thereof can be determined.

### EMBODIMENTS

The invention provides also the following non-limiting embodiments.

Embodiment 1 is a multispecific antibody or antigen binding fragment thereof comprising at least one first antigen-binding region capable of binding specifically to pyroglutamate amyloid-β and a second antigen-binding region capable of binding specifically to transferrin receptor (TfR), wherein:
a. the first antigen-binding region comprises:
   i. a first heavy chain variable region (VH1) comprising heavy chain complementarity determining region 1 (HCDR1), HCDR2, and HCDR3 comprising the amino acid sequences of SEQ ID NOs: 8 or 16, 9 or 17, and 10, respectively; and
   ii. a first light chain variable region (VL1) comprising light chain complementarity determining region 1 (LCDR1), LCDR2, and LCDR3 comprising the amino acid sequences of SEQ ID NOs: 11, 12, and 13, respectively; and
b. the second antigen-binding region comprises:
   i. a second heavy chain variable region (VH2) comprising heavy chain complementarity determining region 1 (HCDR1), HCDR2, and HCDR3 comprising the amino acid sequences of SEQ ID NOs: 1, 2, and 3, respectively; and
   ii. a second light chain variable region (VL2) comprising light chain complementarity determining region 1 (LCDR1), LCDR2, and LCDR3 comprising the amino acid sequences of SEQ ID NOs: 4, 5, and 6, respectively.

Embodiment 2 is the multispecific antibody or antigen binding fragment thereof of embodiment 1, wherein the VH1 comprises an amino acid sequence at least 90% identical to SEQ ID NO:14; and the VL1 comprises an amino acid sequence at least 90% identical to SEQ ID NO:15.

Embodiment 3 is the multispecific antibody or antigen binding fragment thereof of embodiment 1, wherein the VH1 comprises the amino acid sequence of SEQ ID NO:14; and the VL1 comprises the amino acid sequence of SEQ ID NO:15.

Embodiment 4 is the multispecific antibody or antigen binding fragment thereof of any one of embodiments 1-3, wherein the second antigen-binding region comprises a single chain fragment variable (scFv) antibody or antigen binding fragment thereof comprising the VH2 and VL2.

Embodiment 5 is the multispecific antibody or antigen binding fragment thereof of embodiment 4, wherein the scFv comprises an amino acid sequence at least 90% identical to SEQ ID NO:7.

Embodiment 6 is the multispecific antibody or antigen binding fragment thereof of embodiment 5, wherein the scFV comprises the amino acid sequence of SEQ ID NO:7.

Embodiment 7 is the multispecific antibody or antigen binding fragment thereof of any one of embodiments 1-6, wherein the multispecific antibody or antigen binding fragment thereof comprises two first antigen binding regions.

Embodiment 8 is the multispecific antibody or antigen binding fragment thereof of embodiment 7, comprising:
(i) a first heavy chain (HC1) comprising the VH1, a first heavy chain constant region comprising a first Fc region (Fc1), and the scFv
(ii) a second heavy chain (HC2) comprising the VH1 and a second heavy chain constant region comprising a second Fc region (Fc2);
(iii) a first and second light chain (LC) each comprising the VL1 and a light chain constant region.

Embodiment 9 is the multispecific antibody or antigen binding fragment thereof of embodiment 8, wherein the scFv is linked to the carboxy terminus of the first heavy chain constant region via a linker, more particularly a linker comprising the amino acid sequence of SEQ ID NO:27.

Embodiment 10 is the multispecific antibody or antigen binding fragment thereof of embodiment 8 or 9, wherein the Fc1 and Fc2 each comprise one or more heterodimeric mutations, such as a first and a second modified heterodimeric CH3 domains, respectively, as compared to a wild-type Fc region; particularly, the Fc1 comprises amino acid modifications at positions T350, L351, F405, and Y407, and the Fc2 comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat, more particularly, the Fc1 comprises amino acid modifications T350V, L351Y, F405A and Y407V, and the Fc2 comprises amino acid modifications T350V, T366L, K392L and T394W.

Embodiment 11 is the multispecific antibody or antigen binding fragment thereof of any one of embodiments 8-10, wherein the Fc1 and Fc2 each comprise one or more heterodimeric mutations, such as a first and a second modified heterodimeric CH3 domains, respectively, as compared to a wild-type Fc region, wherein the Fc1 comprises amino acid modifications at positions T364, L366, and Y406, and the Fc2 comprises amino acid modifications at positions T365, wherein the amino acid modification at position T364 is T364S; the amino acid modification at position L366 is L366A; the amino acid modification at position Y406 is Y406V; the amino acid modification at position T365 is T365W, and wherein the numbering of amino acid residues is according to the EU index as set forth in Kabat, more particularly, the Fc1 comprises amino acid modifications T364S, L366A, and Y406V, and the Fc2 comprises amino acid modifications T365W.

Embodiment 12 is the multispecific antibody or antigen binding fragment thereof of any one of embodiments 8-11, wherein at least one of the Fc1 and Fc2 comprises one or more mutations that enhance binding of the multispecific antibody or antigen binding fragment thereof to the neonatal Fc receptor (FcRn), preferably the one or more mutations enhance the binding at an acidic pH, more preferably the at least one of the Fc1 and Fc2 has the M252Y/S254T/T256E (YTE) mutations, wherein the numbering of amino acid residues is according to the EU index as set forth in Kabat.

Embodiment 13 is the multispecific antibody or antigen binding fragment thereof of any one of embodiments 8-12, wherein at least one of the Fc1 and Fc2 comprises one or more mutations that reduce or eliminate the effector function, preferably the at least one of the Fc1 and Fc2 has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

Embodiment 14 is a multispecific antibody or antigen binding fragment thereof comprising a first heavy chain comprising an amino acid sequence at least 90% identical to SEQ ID NO:24, and a first light chain comprising an amino acid sequence at least 90% identical to SEQ ID NO:25, a second heavy chain comprising an amino acid sequence at least 90% identical to SEQ ID NO:26, and a second light chain comprising an amino acid sequence at least 90% identical to SEQ ID NO:25.

Embodiment 15 is the multispecific antibody or antigen binding fragment thereof of embodiment 14, wherein the first heavy chain comprises the amino acid sequence of SEQ ID NO:24, the first light chain comprises the amino acid sequence of SEQ ID NO:25, the second heavy chain comprises the amino acid sequence of SEQ ID NO:26, and the second light chain comprises the amino acid sequence of SEQ ID NO:25.

Embodiment 16 is an isolated nucleic acid sequence encoding the multispecific antibody or antigen binding fragment thereof of any one of embodiments 1-16.

Embodiment 17 is a vector comprising the isolated nucleic acid of embodiment 16.

Embodiment 18 is a host cell comprising the isolated nucleic acid of embodiment 16 or the vector of embodiment 17.

Embodiment 19 is a method of producing a multispecific antibody or antigen binding fragment thereof, the method comprising culturing the host cell of embodiment 18 under conditions to produce the multispecific antibody or antigen binding fragment thereof and recovering the multispecific antibody or antigen binding fragment thereof.

Embodiment 20 is a pharmaceutical composition comprising the multispecific antibody or antigen binding fragment thereof of any one of embodiments 1-15 and a pharmaceutically acceptable carrier.

Embodiment 21 is a method of treating or detecting a disorder, preferably a neurological disorder, in a subject in need thereof, comprising administering to the subject the multispecific antibody or antigen-binding fragment of any one of embodiments 1-15, or the pharmaceutical composition of embodiment 20, preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

Embodiment 22 is a method of treating a condition associated with the formation of plaques containing beta-amyloid protein in a subject in need thereof, the method comprising administering the multispecific antibody or antigen binding fragment thereof of any one of embodiments 1-15 or the pharmaceutical composition of embodiment 20 to the subject in need thereof.

Embodiment 23 is the method of embodiment 22 wherein the condition is Alzheimer's disease.

Embodiment 24 is the method of embodiment 22 wherein the condition is selected form the group consisting of dementia associated with Trisomy 21 (Down's Syndrome), diffuse Lewy body disease, inclusion body myositis, cerebral amyloid angiopathy and hereditary cerebral hemorrhage with amyloidosis of the Dutch-type (HCHWA-D).

Embodiment 25 is a method of reducing plaques associated with Alzheimer's disease in a subject in need thereof, the method comprising administering the multispecific antibody or antigen binding fragment thereof of any one of embodiments 1-15 or the pharmaceutical composition of embodiment 20 to the subject in need thereof.

Embodiment 26 is a method of preventing seeding activity of 3pE Aβ in a subject in need thereof, the method comprising administering the multispecific antibody or antigen-binding fragment thereof of any one of embodiments 1-15 or the pharmaceutical composition of embodiment 20 to the subject in need thereof.

Embodiment 27 is a method of producing a pharmaceutical composition comprising the multispecific antibody or antigen-binding fragment thereof of any one of embodiments 1-15, the method comprising combining the multispecific antibody or antigen-binding fragment thereof with a pharmaceutically acceptable carrier to obtain the pharmaceutical composition.

The following examples of the invention are to further illustrate the nature of the invention. It should be understood that the following examples do not limit the invention and the scope of the invention is to be determined by the appended claims.

### EXAMPLES

### Example 1: Creation of Anti-Abeta/Anti-Transferrin Receptor (TfR) Expression Constructs

The cell line development (CLD) process incorporated the use of the ATUM's Leap-In Transposase® technology and the Horizon Chinese Hamster Ovary (CHO) Host cell line. The ATUM Leap-In Transposase® system was engineered from Xenopus tropicalis (western clawed frog). In this system, a synthetic transposon is cloned into a single plasmid containing a selectable marker and the intended biotherapeutic genes between the Leap-In Inverted Terminal Repeats (ITRs). Each gene was placed under control of its own regulatory elements including a promoter and polyadenylation sequence.

The DNA encoding the anti-pyroglutamate amyloid-β/anti-TfR antibody was synthesized and subcloned into the Leap-In Transposase® glutamine synthetase expression vector backbone pD2546ht+_n at ATUM (CA, USA) by Golden Gate Assembly using Type IIS restriction enzyme BsaI and SapI. The plasmid was designated as Plasmid 1 [Light Chain, Heavy Chain 1] and Plasmid 2 [Light Chain, Heavy Chain 2].

The whole plasmids were bi-directional sequence confirmed at NeoGenomics Laboratories (CA, USA).

The primary transcript nucleotide sequence for the Heavy Chain 1, Light Chain, and Heavy Chain 2 genes are listed below. The predicted amino acid sequence for the Heavy Chain 1, Light Chain, and Heavy Chain 2 genes are listed below. Note that the Light Chain gene is identical in Plasmid 1 and 2.

Nucleotide sequence of heavy chain 1 (SEQ ID NO: 18) for Plasmid 1 with signal
peptide underlined:
ATGGCCAGGAAGTCCGCTCTGCTCGCTCTGGCACTTCTGCTTCTGGGATTTGGACCT
GCTTGGGCTCAGGTTCAGCTGGTTCAGTCTGGAGCCGAAGTGAAGAAACCTGGCGC
CTCTGTGAAGGTGTCCTGCAAGGCTTCTGGCCACGTGTTCACCAGCTACGACATGTA
CTGGGTCCGACAGGCTCCTGGACAGGGACTTGAGTGGATCGGCTACATCGACTCCG
ACTCCGGCGACACCTCCTACAACCAGAAATTCAAGGGCAGAGTGACCCTGACCGTG
GACACCTCTACCTCCACCGTGTACATGGAACTGTCCAGCCTGAGATCCGAGGACACC
GCCGTGTACTACTGCGCCTACTACAGATACGCCATGGACTACTGGGGCCAGGGCAC
ACTGGTTACCGTTTCTTCTGCCTCCACCAAGGGCCCATCGGTCTTCCCCCTGGCACCC
TCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGA
CCGTGCCCTCCAGCAGCTTGGGCACCCAGACCTACATCTGCAACGTGAATCACAAGC
CCAGCAACACCAAGGTGGACAAGAAAGTTGAGCCCAAATCTTGTGACAAAACTCAC
ACATGCCCACCGTGCCCAGCACCTGAAGCCGCCGGGGGACCGTCAGTCTTCCTCTTC
CCCCCAAAACCCAAGGACACCCTCTACATCACCCGGGAGCCTGAGGTCACATGCGT
GGTGGTGAGCGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGG
AGTACAAGTGCAAGGTGTCCAACAAAGCCCTCCCAGCCCCCATCGAGAAAACCATC
TCCAAAGCCAAAGGGCAGCCCCGAGAACCACAGGTGTACACCCTGCCCCCATCCCG
GGAGGAGATGACCAAGAACCAGGTCAGCCTGTCCTGCGCCGTCAAAGGCTTCTATC
CCAGCGACATCGCCGTGGAGTGGGAGAGCAATGGGCAGCCGGAGAACAACTACAA
GACCACGCCTCCCGTGCTGGACTCCGACGGCTCCTTCTTCCTCGTGAGCAAGCTCAC
CGTGGACAAGAGCAGATGGCAGCAGGGGAACGTCTTCTCATGCTCCGTGATGCATG
AGGCTCTGCACAACCGGTTCACGCAGAAGTCTCTCTCCCTGTCTCCGGGAAAAGGCG
GAGCTGGTGGTGCTGAGGTTCAGCTGCTCGAGTCTGGCGGAGGATTGGTTCAGCCTG
GCGGCTCTCTGAGACTGTCTTGTGCCGACTCCGGCTTCACCTTCTCCAGCTACGCCAT
GAACTGGGTCCGACAGGCTCCTGGCTGTGGACTGGAATGGGTGTCCGGCATTTCTGG
CTCTGGCGGCCACACCTACTACGCCGATTCTGTGAAGGGCAGATTCACCGTGTCTCG
GGACAACTCCAAGAACACCCTGTACCTGCAGATGAACTCCCTGAGAGCCGAGGACA
CCGCCGTGTACTACTGTGCTAGAGAGGGCTACGACTCCTCCGGCTACAACCCCTTTG
ATTACTGGGGCCAGGGCACCCAAGTGACCGTTTCTTCTGGCGGAGGTTCTGGTGGAT
CTGGCGGATGTCCTCCTTGTGGTGGAAGCGGCGGATCCTACGAGCTGACCCAGCCTC
CTTCCGTGTCTGTGTCTCCTGGCCAGACCGCCTCCATCACCTGTTCTGGCGATAAGCT
GGGCGATAAGTACGCCTCCTGGTATCAGCAGAAGCCCGGCCAGTCTCCTGTGCTGGT
CATCTACCAGGACTCCAAGCGGCCTTCTGGCATCCCTGAGAGATTCTCCGGCTCCAA
CTCCGGCAATACCGCCACACTGACCATCTCTGGCACACAGGCCATGGACGAGGCCG
ACTACTATTGTCAGGCCTGGGACTCCTCCACCGTGGTGTTTGGCTGTGGCACCAAGC
TGACAGTGCTGCAC
Nucleotide sequence of light chain (SEQ ID NO: 19) for Plasmid 1 with signal
peptide underlined:
ATGGCCAGGAAGTCCGCTCTGCTCGCTCTGGCACTTCTGCTTCTGGGATTTGGACCT
GCTTGGGCTGACGTGGTCATGACACAGAGCCCTCTGAGCCTGCCTGTGACATTGGGA
CAGCCTGCCTCCATCTCCTGCAAGTCCTCTCAGTCCCTGCTGGACTCCAGAGCCAAG
ACCTATCTGACCTGGCTGCAGCAGAGGCCTGGCCAGTCTCCTAGAAGGCTGATCTAC
CTGGTGTCCAAGCTGGACTCTGGCGTGCCCGATAGATTCTCCGGCTCTGGCTCTGGC
ACCGACTTCACCCTGAAGATCTCCAGAGTGGAAGCCGAGGACGTGGGCGTGTACTA
CTGTTGGCAGGGCACCCACTTTCCATACACCTTCGGCCAGGGCACAAAGCTGGAAAT
CAAGCGTACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCC
AAAGTACAGTGGAAGGTGGATAACGCCCTCCAATCGGGTAACTCCCAGGAGAGTGT
CACAGAGCAGGACAGCAAGGACAGCACCTACAGCCTCAGCAGCACCCTGACGCTGA
GCAAAGCAGACTACGAGAAACACAAAGTCTACGCCTGCGAAGTCACCCATCAGGGC
CTGAGCTCGCCCGTCACAAAGAGCTTCAACAGGGGAGAGTGT
Nucleotide sequence of heavy chain 2 (SEQ ID NO: 20) for Plasmid 2 with signal
peptide underlined:
ATGGCCAGGAAGTCCGCTCTGCTCGCTCTGGCACTTCTGCTTCTGGGATTTGGACCT
GCTTGGGCTCAGGTTCAGCTGGTTCAGTCTGGAGCCGAAGTGAAGAAACCTGGCGC
CTCTGTGAAGGTGTCCTGCAAGGCTTCTGGCCACGTGTTCACCAGCTACGACATGTA
CTGGGTCCGACAGGCTCCTGGACAGGGACTTGAGTGGATCGGCTACATCGACTCCG
ACTCCGGCGACACCTCCTACAACCAGAAATTCAAGGGCAGAGTGACCCTGACCGTG
GACACCTCTACCTCCACCGTGTACATGGAACTGTCCAGCCTGAGATCCGAGGACACC
GCCGTGTACTACTGCGCCTACTACAGATACGCCATGGACTACTGGGGCCAGGGCAC
ACTGGTTACCGTTTCTTCTGCCTCCACCAAGGGCCCATCGGTCTTCCCCCTGGCACCC
TCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGA
CCGTGCCCTCCAGCAGCTTGGGCACCCAGACCTACATCTGCAACGTGAATCACAAGC
CCAGCAACACCAAGGTGGACAAGAAAGTTGAGCCCAAATCTTGTGACAAAACTCAC
ACATGCCCACCGTGCCCAGCACCTGAAGCCGCCGGGGGACCGTCAGTCTTCCTCTTC
CCCCCAAAACCCAAGGACACCCTCTACATCACCCGGGAGCCTGAGGTCACATGCGT
GGTGGTGAGCGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGG
AGTACAAGTGCAAGGTGTCGAACAAAGCCCTCCCAGCCCCCATCGAGAAAACCATC
TCCAAAGCCAAAGGGCAGCCCCGAGAACCACAGGTGTACACCCTGCCCCCATCCCG
GGAGGAGATGACCAAGAACCAGGTCAGCCTGTGGTGCCTGGTCAAAGGCTTCTATC
CCAGCGACATCGCCGTGGAGTGGGAGAGCAATGGGCAGCCGGAGAACAACTACAA
GACCACGCCTCCCGTGCTGGACTCCGACGGCTCCTTCTTCCTCTACAGCAAGCTCAC
CGTGGACAAGAGCAGATGGCAGCAGGGGAACGTCTTCTCATGCTCCGTGATGCATG
AGGCTCTGCACAACCACTACACGCAGAAGTCTCTCTCCCTGTCTCCGGGAAAA
Amino acid sequence of heavy chain 1 (SEQ ID NO: 21) for Plasmid 1 with signal
peptide underlined. The Gln (Q) residue at position 1 of the heavy chain 1
constitutes the N-terminus of the mature chain.
MARKSALLALALLLLGFGPAWAQVQLVQSGAEVKKPGASVKVSCKASGHVFTSYDMY
WVRQAPGQGLEWIGYIDSDSGDTSYNQKFKGRVTLTVDTSTSTVYMELSSLRSEDTAV
YYCAYYRYAMDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLYITREPEVTCVVVSVSHEDPE
VKFNWYVDGVEVHNAKTKPREEQYNSTYRVVSVLTVLHQDWLNGKEYKCKVSNKAL
PAPIEKTISKAKGQPREPQVYTLPPSREEMTKNQVSLSCAVKGFYPSDIAVEWESNGQPE
NNYKTTPPVLDSDGSFFLVSKLTVDKSRWQQGNVFSCSVMHEALHNRFTQKSLSLSPGK
GGAGGAEVQLLESGGGLVQPGGSLRLSCADSGFTFSSYAMNWVRQAPGCGLEWVSGIS
GSGGHTYYADSVKGRFTVSRDNSKNTLYLQMNSLRAEDTAVYYCAREGYDSSGYNPF
DYWGQGTQVTVSSGGGSGGSGGCPPCGGSGGSYELTQPPSVSVSPGQTASITCSGDKLG
DKYASWYQQKPGQSPVLVIYQDSKRPSGIPERFSGSNSGNTATLTISGTQAMDEADYYC
QAWDSSTVVFGCGTKLTVLH
Amino acid sequence of light chain (SEQ ID NO: 22) for Plasmid 1 with signal
peptide underlined. The Asp (D) residue at position 1 of the light chain
constitutes the N-terminus of the mature chain.
MARKSALLALALLLLGFGPAWADVVMTQSPLSLPVTLGQPASISCKSSQSLLDSRAKTY
LTWLQQRPGQSPRRLIYLVSKLDSGVPDRFSGSGSGTDFTLKISRVEAEDVGVYYCWQG
THEPYTFGQGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSEN
RGEC
Amino acid sequence of heavy chain 2 (SEQ ID NO: 23) for Plasmid 2 with signal
peptide underlined. The Gln (Q) residue at position 1 of the heavy chain
constitutes the N-terminus of the mature chain.
MARKSALLALALLLLGFGPAWAQVQLVQSGAEVKKPGASVKVSCKASGHVFTSYDMY
WVRQAPGQGLEWIGYIDSDSGDTSYNQKFKGRVTLTVDTSTSTVYMELSSLRSEDTAV
YYCAYYRYAMDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLYITREPEVTCVVVSVSHEDPE
VKFNWYVDGVEVHNAKTKPREEQYNSTYRVVSVLTVLHQDWLNGKEYKCKVSNKAL
PAPIEKTISKAKGQPREPQVYTLPPSREEMTKNQVSLWCLVKGFYPSDIAVEWESNGQPE
NNYKTTPPVLDSDGSFFLYSKLTVDKSRWQQGNVFSCSVMHEALHNHYTQKSLSLSPG
K
Mature amino acid sequence of heavy chain 1 (SEQ ID NO: 24) for Plasmid 1:
QVQLVQSGAEVKKPGASVKVSCKASGHVFTSYDMYWVRQAPGQGLEWIGYIDSDSGD
TSYNQKFKGRVTLTVDTSTSTVYMELSSLRSEDTAVYYCAYYRYAMDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEAA
GGPSVFLFPPKPKDTLYITREPEVTCVVVSVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLSCAVKGFYPSDIAVEWESNGQPENNYKTTPPVLDSDGSFFL
VSKLTVDKSRWQQGNVFSCSVMHEALHNRFTQKSLSLSPGKGGAGGAEVQLLESGGGL
VQPGGSLRLSCADSGFTFSSYAMNWVRQAPGCGLEWVSGISGSGGHTYYADSVKGRFT
VSRDNSKNTLYLQMNSLRAEDTAVYYCAREGYDSSGYNPFDYWGQGTQVTVSSGGGS
GGSGGCPPCGGSGGSYELTQPPSVSVSPGQTASITCSGDKLGDKYASWYQQKPGQSPVL
VIYQDSKRPSGIPERFSGSNSGNTATLTISGTQAMDEADYYCQAWDSSTVVFGCGTKLT
VLH
Mature amino acid sequence of light chain 1 (SEQ ID NO: 25) for Plasmid 1:
DVVMTQSPLSLPVTLGQPASISCKSSQSLLDSRAKTYLTWLQQRPGQSPRRLIYLVSKLD
SGVPDRFSGSGSGTDFTLKISRVEAEDVGVYYCWQGTHEPYTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Mature amino acid sequence of heavy chain 2 (SEQ ID NO: 26) for Plasmid 2:
QVQLVQSGAEVKKPGASVKVSCKASGHVFTSYDMYWVRQAPGQGLEWIGYIDSDSGD
TSYNQKFKGRVTLTVDTSTSTVYMELSSLRSEDTAVYYCAYYRYAMDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEAA
GGPSVFLFPPKPKDTLYITREPEVTCVVVSVSHEDPEVKFNWYVDGVEVHNAKTKPREE
QYNSTYRVVSVLTVLHQDWLNGKEYKCKVSNKALPAPIEKTISKAKGQPREPQVYTLPP
SREEMTKNQVSLWCLVKGFYPSDIAVEWESNGQPENNYKTTPPVLDSDGSFFLYSKLTV
DKSRWQQGNVFSCSVMHEALHNHYTQKSLSLSPGK
Cell lines expressing the anti-pyroglutamate amyloid-β/anti-TfR constructs were
generated.
Example 2: Binding characterization by Surface Plasmon Resonance
The binding interaction of test articles against recombinant β-amyloid peptide (3pE-28)
and human transferrin receptor (TfR; TFRW2) are studied by SPR using a Biacore 8k
instrument at 25° C. with pH 7.4 or pH 5.0 buffer, supplemented with 3 mM EDTA, and
0.05% Tween 20. Briefly, a biosensor surface is prepared by coupling anti-human IgG
Fcγ-fragment specific antibody to the surface of a C1 sensor chip using vendor
recommended protocol for amine-coupling chemistry (>400 response units (RU)). The
coupling buffer is 10 mM sodium acetate, pH 4.5. The test articles are diluted in the
running buffer and injected over the anti-human IgG to obtain enough capture to
enable detection of antigen binding. Capture of test articles is followed

by the injection of the three recombinant antigens at different concentration series in single-cycle kinetics mode (300 nM to 11.1 nM series at 3-fold dilutions for TfR, 12 nM to 0.8 nM series at 2-fold dilutions for 3pE-28). The association and dissociation are monitored for 60 min (for 3pE-28 antigens) and for 2 minutes and 5 minutes for TfR antigen at 50 μL/min flow rate. The dissociation profile of TfR is measured at both pH 7.4 and pH 5.0. Regeneration of the sensor surface is performed with 0.85% H₃PO₄. The binding sensorgrams are fitted using to a 1:1 Langmuir binding model to obtain on-rates, off-rates, and affinities. The parental bivalent antibody against β-amyloid was included as control.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A multispecific antibody or antigen binding fragment thereof comprising at least one first antigen-binding region capable of binding specifically to pyroglutamate amyloid and a second antigen-binding region capable of binding specifically to transferrin receptor (TfR), wherein:
a. the first antigen-binding region comprises:
i. a first heavy chain variable region (VH1) comprising heavy chain complementarity determining region 1 (HCDR1), HCDR2, and HCDR3 comprising the amino acid sequences of SEQ ID NOs: 8 or 16, 9 or 17, and 10, respectively; and
ii. a first light chain variable region (VL1) comprising light chain complementarity determining region 1 (LCDR1), LCDR2, and LCDR3 comprising the amino acid sequences of SEQ ID NOs: 11, 12, and 13, respectively; and
b. the second antigen-binding region comprises:
i. a second heavy chain variable region (VH2) comprising heavy chain complementarity determining region 1 (HCDR1), HCDR2, and HCDR3 comprising the amino acid sequences of SEQ ID NOs: 1, 2, and 3, respectively; and
ii. a second light chain variable region (VL2) comprising light chain complementarity determining region 1 (LCDR1), LCDR2, and LCDR3 comprising the amino acid sequences of SEQ ID NOs: 4, 5, and 6, respectively.

2. The multispecific antibody or antigen binding fragment thereof of claim 1, wherein the VH1 comprises an amino acid sequence at least 90% identical to SEQ ID NO:14; and the VL1 comprises an amino acid sequence at least 90% identical to SEQ ID NO:15.

3. The multispecific antibody or antigen binding fragment thereof of claim 1, wherein the VH1 comprises the amino acid sequence of SEQ ID NO:14; and the VL1 comprises the amino acid sequence of SEQ ID NO:15.

4. The multispecific antibody or antigen binding fragment thereof of claim 1, wherein the second antigen-binding region comprises a single chain fragment variable (scFv) antibody or antigen binding fragment thereof comprising the VH2 and VL2.

5. The multispecific antibody or antigen binding fragment thereof of claim 4, wherein the scFv comprises an amino acid sequence at least 90% identical to SEQ ID NO:7.

6. The multispecific antibody or antigen binding fragment thereof of claim 5, wherein the scFV comprises the amino acid sequence of SEQ ID NO:7.

7. The multispecific antibody or antigen binding fragment thereof of claim 1, wherein the multispecific antibody or antigen binding fragment thereof comprises two first antigen binding regions.

8. The multispecific antibody or antigen binding fragment thereof of claim 7, comprising:
(j) a first heavy chain (HC1) comprising the VH1, a first heavy chain constant region comprising a first Fc region (Fc1), and the scFv
(iv) a second heavy chain (HC2) comprising the VH1 and a second heavy chain constant region comprising a second Fc region (Fc2);
(v) a first and second light chain (LC) each comprising the VL1 and a light chain constant region.

9. The multispecific antibody or antigen binding fragment thereof of claim 8, wherein the scFv is linked to the carboxy terminus of the first heavy chain constant region via a linker, more particularly a linker comprising the amino acid sequence of SEQ ID NO:27.

10. The multispecific antibody or antigen binding fragment thereof of claim 8, wherein the Fc1 and Fc2 each comprise one or more heterodimeric mutations, such as a first and a second modified heterodimeric CH3 domains, respectively, as compared to a wild-type Fc region; particularly, the Fc1 comprises amino acid modifications at positions T350, L351, F405, and Y407, and the Fc2 comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat, more particularly, the Fc1 comprises amino acid modifications T350V, L351Y, F405A and Y407V, and the Fc2 comprises amino acid modifications T350V, T366L, K392L and T394W.

11. The multispecific antibody or antigen binding fragment thereof of claim 8, wherein at least one of the Fc1 and Fc2 comprises one or more mutations that enhance binding of the multispecific antibody or antigen binding fragment thereof to the neonatal Fc receptor (RcRn), preferably the one or more mutations enhance the binding at an acidic pH, more preferably the at least one of the Fc1 and Fc2 has the M252Y/S254T/T256E (YTE) mutations, wherein the numbering of amino acid residues is according to the EU index as set forth in Kabat.

12. The multispecific antibody or antigen binding fragment thereof of claim 8, wherein at least one of the Fc1 and Fc2 comprises one or more mutations that reduce or eliminate the effector function, preferably the at least one of the Fc1 and Fc2 has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

13. A multispecific antibody or antigen binding fragment thereof comprising a first heavy chain comprising an amino acid sequence at least 90% identical to SEQ ID NO:24, and a first light chain comprising an amino acid sequence at least 90% identical to SEQ ID NO:25; a second heavy chain comprising an amino acid sequence at least 90% identical to SEQ ID NO:26, and a second light chain comprising an amino acid sequence at least 90% identical to SEQ ID NO:25.

14. The multispecific antibody or antigen binding fragment thereof of claim 13, wherein the first heavy chain comprises the amino acid sequence of SEQ ID NO:24, the first light chain comprises the amino acid sequence of SEQ ID NO:25; the second heavy chain comprises the amino acid sequence of SEQ ID NO:26, and the second light chain comprises the amino acid sequence of SEQ ID NO:25.

15. An isolated nucleic acid sequence encoding the multispecific antibody or antigen binding fragment thereof claim 1.

16. A vector comprising the isolated nucleic acid of claim 15.

17. A host cell comprising the isolated nucleic acid of claim 15 or the vector of claim 16.

18. A method of producing a multispecific antibody or antigen binding fragment thereof, the method comprising culturing the host cell of claim 17 under conditions to produce the multispecific antibody or antigen binding fragment thereof and recovering the multispecific antibody or antigen binding fragment thereof.

19. A pharmaceutical composition comprising the multispecific antibody or antigen binding fragment thereof of claim 1 and a pharmaceutically acceptable carrier.

20. A method of treating or detecting a disorder, preferably a neurological disorder, in a subject in need thereof, comprising administering to the subject the multispecific antibody or antigen-binding fragment of claim 1, preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

21. A method of treating a condition associated with the formation of plaques containing beta-amyloid protein in a subject in need thereof, the method comprising administering the multispecific antibody or antigen binding fragment thereof of claim 1 to the subject in need thereof.

22. The method of claim 21 wherein the condition is Alzheimer's disease.

23. The method of claim 21 wherein the condition is selected form the group consisting of dementia associated with Trisomy 21 (Down's Syndrome), diffuse Lewy body disease, inclusion body myositis, cerebral amyloid angiopathy and hereditary cerebral hemorrhage with amyloidosis of the Dutch-type (HCHWA-D).

24. A method of reducing plaques associated with Alzheimer's disease in a subject in need thereof, the method comprising administering the multispecific antibody or antigen binding fragment thereof of claim 1 to the subject in need thereof.

25. A method of preventing seeding activity of 3pE Aβ in a subject in need thereof, the method comprising administering the multispecific antibody or antigen-binding fragment thereof of claim 1 to the subject in need thereof.

26. A method of producing a pharmaceutical composition comprising the multispecific antibody or antigen-binding fragment thereof of claim 1, the method comprising combining the multispecific antibody or antigen-binding fragment thereof with a pharmaceutically acceptable carrier to obtain the pharmaceutical composition.
